(19) United States
(12) Patent Application Publication
(10) Pub. No.: US 2017/0261505 A1
(43) Pub. Date: Sep. 14, 2017

(54) METHOD AND KIT FOR THE PREDICTIVE PROGNOSIS OF RESPONSIVENESS TO TREATMENTS OF AUTOIMMUNE DISEASES

(71) Applicant: UNIVERSITA' DEGLI STUDI DI ROMA "LA SAPIENZA", Roma (IT)

(72) Inventor: Vincenzo BARNABA, Roma (IT)

(21) Appl. No.: 15/511,624

(22) PCT Filed: Sep. 7, 2015
(86) PCT No.: PCT/IB2015/056829

§ 371 Date: Mar. 15, 2017

(30) Foreign Application Priority Data
Sep. 16, 2014 (IT) RM2014A000524

(51) Int. Cl. G01N 33/564

(52) U.S. Cl. CPC G01N 33/564; G01N 2800/102; G01N 2800/52; G01N 2800/24; G01N 2800/104; G01N 2800/065

(57) ABSTRACT

The present invention relates to the use of one or more MHC class I molecules dextramers (Dextramers®) associated with peptides corresponding to Apoptotic Epitopes of human CD8⁺ T cells for the predictive prognosis of responsiveness or non-responsiveness to treatments and/or for monitoring the therapeutic effectiveness of treatments with biological medicaments that block and/or inhibit TNF, and/or biological medicaments that block and/or inhibit cytokines or cytokine receptors and/or biological medicaments against B cells and/or biological medicaments that inhibit T cell co-stimulation in patients affected by autoimmune diseases, together with methods and kits for said predictive prognosis.

The present invention relates to the use of one or more MHC class I molecules dextramers (Dextramers®) associated with peptides corresponding to Apoptotic Epitopes of human T lymphocytes (or cells) for the predictive prognosis of responsiveness or non-responsiveness to treatments and/or for monitoring the therapeutic effectiveness of said treatments with biological medicaments that block and/or inhibit TNF, and/or biological medicaments that block and/or inhibit cytokines or cytokine receptors and/or biological medicaments against B cells and/or biological medicaments that inhibit T cell co-stimulation in patients affected by autoimmune diseases, together with methods and kits for said predictive prognosis.

### STATE OF THE PRIOR ART

In previous studies, the Authors of the present invention had demonstrated that the proteome of apoptotic T cells includes cellular proteins cleaved by caspases (enzymes enabling cell death by protein and DNA fragmentation): fragments of: actin cytoplasmic 1 [ACTB], heterogeneous nuclear ribonucleoprotein [ROK], lamin B1 [LAM1], non-muscle myosin heavy chain 9 [MYH9], vimentin [VIME], proteasome component C2 [PSA1], rho GDP dissociation inhibitor 2 [GDIS], and 60S acidic ribosomal protein P2 [RLA] (Propato et al, Nat Med 2001; Rawson et al, Nat Med 2007). The Authors demonstrated that caspases, in apoptotic cells, generate from “long-lived” proteins that are strictly anchored to cellular structures such as the cytoskeleton, and that these antigenic fragments are efficiently processed, and cross-presented by dendritic cells (DCs) to an enormous repertoire of CD8-specific T lymphocytes (or cells) with pro-inflammatory potential. In addition, apoptotic cells derived from activated T lymphocytes retain the expression of CD40 ligand expressed by DCs, which can thus become highly priming for T cells. In chronic inflammatory diseases, such as HIV, or hepatitis C virus (HCV) or hepatitis B virus (HBV), the proportion of circulating apoptotic lymphocytes proves to be correlated with the frequency of CD8 T lymphocytes specific for the peptides (epitopes) derived from the apoptotic antigens.

The Inventors further demonstrated that the frequencies of such CD8 T lymphocytes specific to Apoptotic Epitopes correlate with disease activity in such infections (Franceschini et al, Plos Pathog 2012, and PCT WO2012/159993 “Method to prognose viral infections by measuring T cell responses or autoantibodies to apoptotic epitopes”.

Autoimmune diseases are diseases deriving from an anomalous immune response of the body against substances and tissues normally present in the body (autoimmunity). This anomalous response may be limited to certain organs or may be, e.g., against a specific tissue. The treatment of autoimmune diseases is generally performed with immunosuppressive medicaments reducing the immune response. At least three types of medicaments utilized in autoimmune disease therapy are known, i.e. synthetic medicaments, biological medicaments and biosimilar medicaments.

Biological medicaments are, e.g., tumor necrosis factor (TNF) inhibitors, T cell co-stimulation inhibitors, anti-B cell agents, cytokine inhibitors or cytokine receptor inhibitors.

Autoimmune diseases are moreover characterised by the presence of the phenomenon of chronic immune activation.

Autoimmune diseases represent about 20% of human pathologies and are conditioned by strong morbidity, co-morbidity and very costly therapies, of unforeseeable outcome, which make their management at a National Health System level very expensive and difficult.

The discovery of novel biomarkers predicting the prognosis of such pathologies and enabling to discriminate between patients that will be responsive or non-responsive to certain therapies, such as, e.g., the costly biological therapies, represents a great result to be obtained in this field of medicine.

### SUMMARY OF THE INVENTION

The Authors of the present invention discovered that the responses of CD8 T lymphocytes specific to Apoptotic Epitopes are predictive of disease progression and of response to therapies with certain biological medicaments.

The Authors of the invention applied to samples taken from autoimmune disease patients the methodology used in PCT Application WO2012/159993 (enzyme-linked immunospot assay [ELISPOT]) and the data obtained showed that the number of CD8 T lymphocytes specific to Apoptotic Epitopes was greater in samples of individuals affected by autoimmune diseases than in healthy ones, but that there was no difference between the samples from individuals responsive to therapies with certain biological medicaments and samples from non-responsive individuals. Despite the data obtained, the Authors of the present invention have amazingly discovered that the use of different technologies enables to detect a different percentage of CD8 T lymphocytes specific to Apoptotic Epitopes, and different populations of these lymphocytes, in samples from individuals responsive to therapies with biological medicaments and samples from non-responsive individuals.

The Authors, in fact, found that the frequencies of CD8 T lymphocytes specific against Apoptotic Epitopes are significantly higher in patients affected by autoimmune diseases than in healthy control subjects, and that these frequencies are correlated with disease activity. In addition, the Authors of the present invention found (as may be seen in the Experimental Section) that such responses proved significantly higher in patients that would then have responded (responders (R)) to a therapy with above-described biological medicaments, than in non-responders (NR). This data, together with the finding that no clinical criteria was capable of discriminating R from NR at a subsequent therapy regimen, teaches that the determining of the frequencies of CD8 T lymphocytes specific to Apoptotic Epitopes configures as a single biomarker predicting response to therapy. In other words, in patients with autoimmune diseases, the significant increase of responses of CD8 T lymphocytes specific to Apoptotic Epitopes in R than in NR proves to be a valid and important biomarker of prognosis and prediction of response to therapy, as it is higher before the start of the same therapy.

Therefore, the determining of responses of CD8 T lymphocytes (T cells) specific to Apoptotic Epitopes configures both as biomarker of disease activity and as biomarker capable of predicting the response to therapy, and therefore a “Therapy Economy” marker, a term defining herein the concept that, by using the biomarkers of the invention, patients that will respond to the various therapies can be selected, with a great economical advantage as to care expenses and with great advantage to the patient, who will avoid useless therapies. The present invention, therefore, defines the first biomarker predictive of disease evolution, of the course of protective effects of anti-inflammatory therapies, and of prognosis of autoimmune diseases and their sequelae.

The present invention therefore relates to:
the use of one or more MHC class I molecules dextramers (Dextramers®) associated with peptides corresponding to Apoptotic Epitopes of human CD8⁺ T cells for the predictive prognosis of responsiveness or non-responsiveness to treatments and/or for monitoring the therapeutic effectiveness of said treatments with biological medicaments that block and/or inhibit TNF, and/or biological medicaments that block and/or inhibit cytokines or cytokine receptors and/or biological medicaments against B cells and/or biological medicaments that inhibit T cell co-stimulation in patients affected by autoimmune diseases;
an ex vivo method for the predictive prognosis of responsiveness or non-responsiveness to treatments with biological medicaments that block and/or inhibit TNF, and/or biological medicaments that block and/or inhibit cytokines or cytokine receptors and/or biological medicaments against B cells and/or biological medicaments that inhibit T cell co-stimulation in patients affected by autoimmune diseases, comprising the following steps:
a. contacting a biological sample to be analysed comprising CD8⁺ T cells and a control biological sample comprising CD8⁺ T cells representative of patients affected by autoimmune diseases that are responsive or non-responsive to said treatments, with one or more dextramers (Dextramers®) of MHC class I molecules associated with peptides corresponding to Apoptotic Epitopes of human CD8⁺ T cells;
b. quantifying the number of CD8⁺ T cells specifically binding said one or more dextramers in each sample;
c. comparing the amount of CD8⁺ T cells specifically binding said one or more dextramers in the analysed samples and predicting the responsiveness or non-responsiveness of the patient associated with said biological sample to be analysed to said treatments on the basis of the amount of CD8⁺ T cells specifically binding said one or more dextramers; p0 an ex vivo method for the predictive prognosis of responsiveness or non-responsiveness to treatments with biological medicaments that block and/or inhibit TNF, and/or biological medicaments that block and/or inhibit cytokines or cytokine receptors and/or biological medicaments against B cells and/or biological medicaments that inhibit T cell co-stimulation in patients affected by autoimmune diseases, comprising the following steps:
a. contacting a biological sample to be analysed comprising CD8⁺ T cells with one or more dextramers (Dextramers®) of MHC class I molecules associated with peptides corresponding to Apoptotic Epitopes of CD8⁺ T cells; b. quantifying the number of CD8⁺ T cells specifically binding said one or more dextramers in the sample, wherein the presence of a percent of CD8⁺ T cells specifically binding said dextramers against the total number of CD8⁺ T cells ≧0.5% is predictive of responsiveness to said treatments;
an ex vivo method for the predictive prognosis of responsiveness or non-responsiveness to treatments with biological medicaments that block and/or inhibit TNF, and/or biological medicaments that block and/or inhibit cytokines or cytokine receptors and/or biological medicaments against B cells and/or biological medicaments that inhibit T cell co-stimulation of patients affected by autoimmune diseases, comprising the following steps:
a. contacting a biological sample to be analysed comprising CD8⁺ T cells with one or more dextramers peptides (Dextramers®) of MHC class I molecules associated with peptides corresponding to Apoptotic Epitopes of human CD8⁺ T cells;
b. quantifying the number of CD8⁺ T cells specifically binding said one or more dextramers in the sample, wherein the presence of a percent of CD8⁺ T cells specifically binding said dextramers against the total number of CD8⁺ T cells ≧0.235% has a predictive value of a 78% responsiveness to said treatments whereas a percent <0.235% has a predictive value of a 75% non-responsiveness to said treatments;
an ex vivo method for monitoring the therapeutic effectiveness of treatments with biological medicaments that block and/or inhibit TNFα and/or biological medicaments that block and/or inhibit cytokines or cytokine receptors and/or biological medicaments against B cells and/or biological medicaments that inhibit T cell co-stimulation in a patient affected by autoimmune disease, comprising the following steps:
a. contacting biological samples comprising CD8⁺ T cells obtained in subsequent moments of time before and during said treatments with one or more dextramers (Dextramers®) of MHC class I molecules associated with peptides corresponding to Apoptotic Epitopes of human CD8⁺ T cells; b) quantifying the number of CD8⁺ T cells specifically binding said one or more dextramers in each sample;
c. evaluating the variation of said percent of CD8⁺ T cells specifically binding said one or more dextramers in said samples in time,
wherein a decrease in the percent of CD8⁺ T cells bound by said one or more dextramers in said samples in time indicates an effective therapy;
a kit for the predictive prognosis of the responsiveness to treatment of one or more diseases with biological medicaments that block and/or inhibit TNFα and/or biological medicaments that block and/or inhibit cytokines or cytokine receptors and/or biological medicaments against B cells and/or biological medicaments that inhibit T cell co-stimulation, and/or for monitoring the effectiveness of said treatment in responsive patients, comprising one or more MHC class I molecules dextramers (Dextramers®) associated with peptides corresponding to Apoptotic Epitopes of human CD8⁺ T cells in one or more aliquots,
one or more aliquots of a control sample representative of healthy individuals, one or more aliquots of a sample representative of individuals affected by an autoimmune disease that are responsive to said treatments and one or more aliquots of a sample representative of individuals affected by said autoimmune disease that are non-responsive to said treatments.

### DETAILED DESCRIPTION OF THE FIGURES

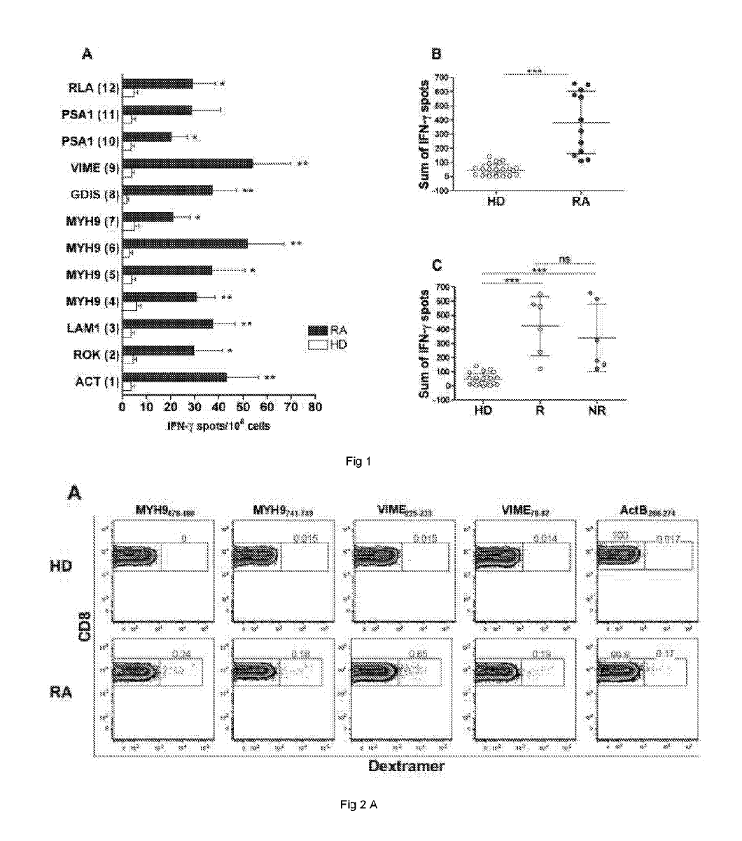

FIG. 1 CD8⁺ T cell multispecificity to AE in HDs and RA patients. (A) Mean number of IFN-γ spots formed by fresh CD8⁺ T_{EM} cells (by ELISPOT assay) in response to 12 pools of AE (see Table 1) in 12 HLA-A2⁺ patients with RA or 24 HLA-A2⁺ healthy donors (HD). (B) Sum of IFN-γ spots formed by fresh CD8⁺ T_{EM} cells in response to all pools (see Table 1) of AE in the single patients or HD. (C) Sum of IFN-γ spots formed by fresh CD8⁺ T_{EM} cells in response to all pools (see Table 1) of AE in the single R, NR, or HD. Statistical analysis was performed with the Mann-Whitney test. *p<0,01; **p<0,001; ***p<0,0001. ns=not significant. The symbols for the proteins from which the peptides derive, i.e., RLA2; PSA1; VIME; GDIS; MYH9; LAM1; ROK; correspond to Apoptotic Epitopes peptides derived from the proteins as described below:
ACTB=Actin cytoplasmic 1 (reference sequence having accession number P02570);
ROK=heterogeneous nuclear ribonucleoprotein k (reference sequence having accession number Q07244);
LAM1=lamin B1 (reference sequence having accession number P20700)
MYH9=non-muscle myosin, heavy chain 9 (reference sequence having accession number P35579)
VIME=vimentin (reference sequence having accession number P08670)
PSA1=proteasome component C2 (reference sequence having accession number P25786)
GDIS=rho GDP-dissociation inhibitor 2 (reference sequence having accession number P52566)
RLA2=60S acidic ribosomal protein P2 (reference sequence having accession number P05387).

ACT B=Actin cytoplasmic 1

Numbers in parentheses correspond to the pool number shown in Table 1 below.

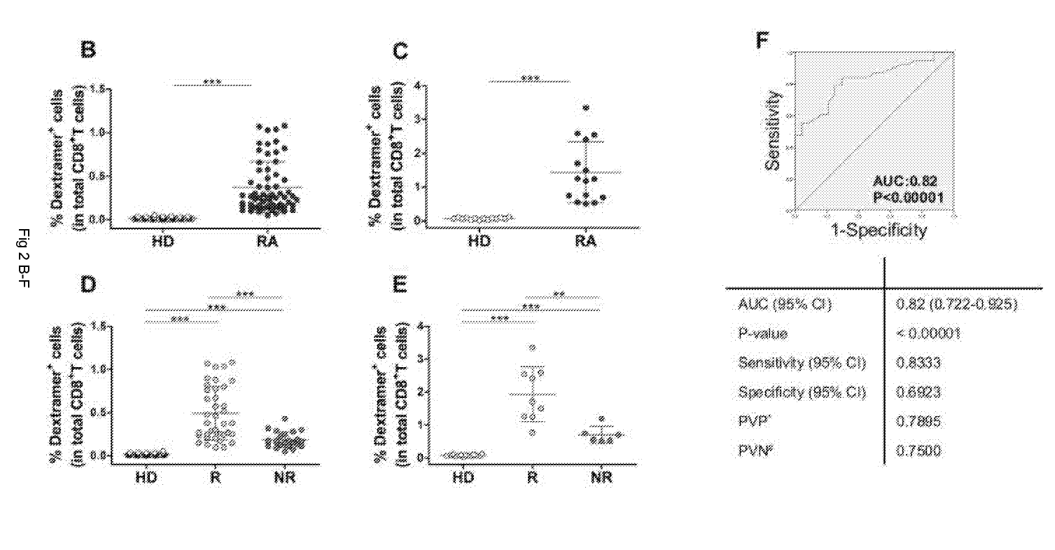

FIG. 2. AE-specific CD8⁺ T cells predict RA patients who will benefit or not from TNF-α inhibitor therapy.

(A) Representative flow cytometry analysis of dextramer®⁺ CD8⁺ T cells specific to Apoptotic Epitopes in an RA patient and an HD. PBMCs were stained with a PeCy7-labeled mAb to CD8, APC-labeled-HLA-A*0201 dextramers® expressing the indicated AE, and the dump channel APC-Cy7-labeled reagents so as to exclude CD4⁺ T cells, monocytes, B cells, NK cells and dead cells from the analysis. Zebra plot analyses show the percentage of dextramer®⁺ CD8⁺ T cells. The percentage of cells is indicated in the appropriate quadrant. (B) Percentage of dextramer®⁺ CD8⁺ T cells specific to a single epitope in 14 HLA-A2⁺ HD and in 15 HLA-A2⁺ patients (each symbol represents the percentage of dextramer®⁺ CD8⁺ T cells specific to a single epitope in HD or patients). (C) Sum of the percentages of all dextramer®⁺ CD8⁺ T cells detected in the single HD or patient (each symbol represents the sum of percentages of the 5 dextramers® tested in the single individual). (D). Percentage of dextramer®⁺ CD8⁺ T cells specific to a single epitope in 14 HLA-A2⁺ HD, 9 HLA-A2⁺ R to TNF-α inhibitor therapy, and 6 HLA-A2⁺ NR (each symbol represents the percentage of dextramer®⁺ CD8⁺ T cells specific to a single epitope in HD or patients). (E) Sum of the percentages of all dextramer®⁺ CD8⁺ T cells detected in the single HD, R, and NR (each symbol represents the sum of percentages of the 5 dextramers® tested in the single individual). Statistical analysis was performed with the Mann-Whitney test. **p<0,001; ***p<0,0001. (F) ROC (Receiver-operator characteristic) curve analyses for R and NR RA patients.

AUC=area under receiver-operator characteristic curve.

*=Predictive value of positive test.

#=Predictive value of negative test.

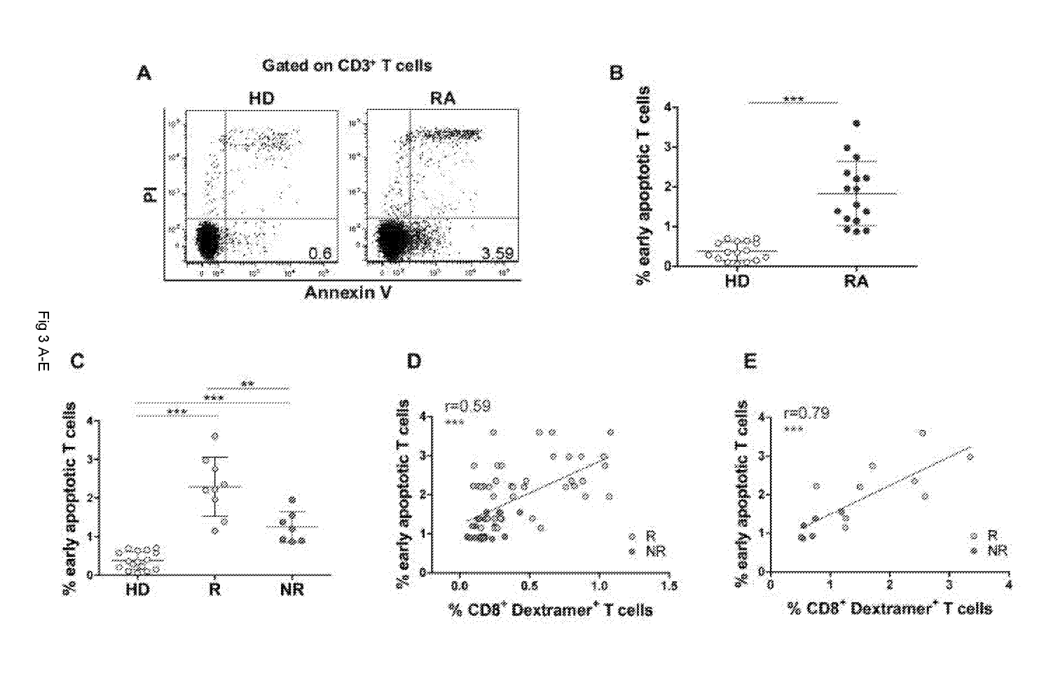

FIG. 3. AE-specific CD8⁺ T cells correlate with circulating apoptotic T cells in RA patients. (A) Representative flow cytometry analysis of double-stained AnnexinV/P1⁺ T cells in CD3⁺ T cells, from a HD or an RA patient. The percentage of cells is indicated in the appropriate quadrant. (B) Percentage of early (Annexin V) apoptotic T cells in 16 HD e 16 RA patients studied. Statistical analysis was performed with the Mann-Whitney test. ***p<0,0001. (C) Percentage of early (Annexin V) apoptotic T cells in all HD and RA patients (9 R or 7 NR to TNF-α inhibitor therapy) studied. Statistical analysis was performed with the Mann-Whitney test. **p<0,001; ***p<0,0001. (D) Correlation between percentage of early (Annexin V) apoptotic T cells and percentage of all Dextramer®⁺ CD8⁺ T cells specific to a single epitope from R or NR (each symbol represents Dextramer®⁺ CD8⁺ T cells specific to a single AE) (Spearman correlation analysis). (E) Correlation between percentage of early (Annexin V) apoptotic T cells and the sum of percentages of Dextramer®⁺ CD8⁺ T cells specific to a single epitope from the single R or NR (each symbol represents a single patient) (Spearman correlation analysis).

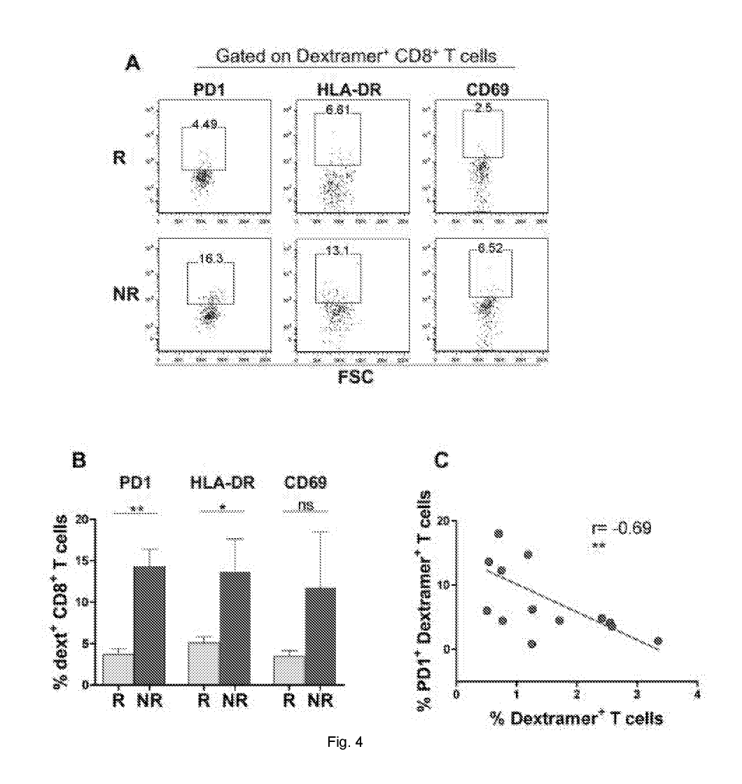

FIG. 4. AE-specific CD8⁺ T cells expressing PD-1 are significantly more represented in NR than in R, and inversely correlate with the frequencies of their total counterparts.

(A) Representative flow cytometry analysis of PD-1, HLA-DR and CD69 in AE-specific dextramer®⁺ CD8⁺ T cells from a R and a NR. (B) Percentage of AE-specific dextramer®⁺ CD8⁺ T cells expressing PD-1, HLA-DR, or CD69 in all R and NR studied. Statistical analysis was performed with the Mann-Whitney test. *p<0,01; **p<0,001; ns=not significant. (C) Correlation between AE-specific dextramer®⁺ CD8⁺ T cells expressing PD-1 and total AE-specific dextramer®⁺ CD8⁺ T cells (Spearman correlation analysis). **p<0,001.

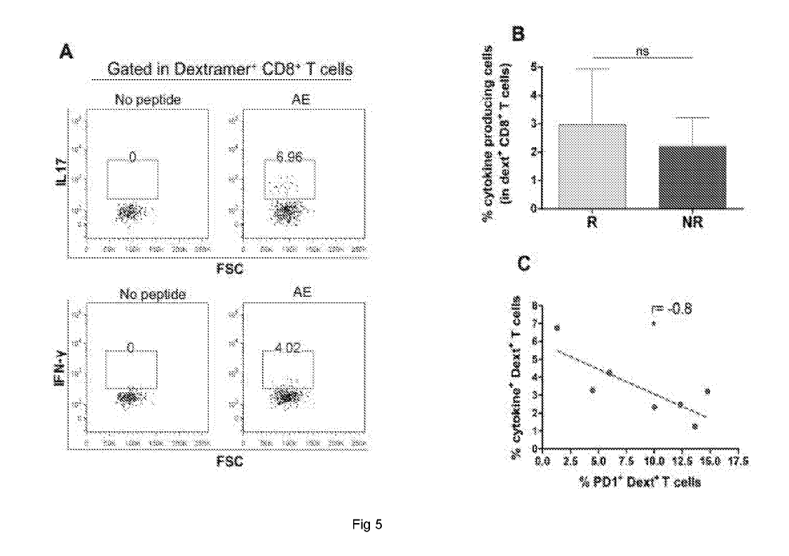

FIG. 5 AE-specific dextramer®⁺ CD8⁺ T cells specifically producing inflammatory cytokines inversely correlate with PD-1⁺ dextramer®⁺CD8⁺ T cells.

(A) Representative flow cytometry analyses of cells producing IL-17 or IFN-γ in Dextramer®⁺ CD8⁺ T cells in response to a relevant AE pool (5 peptides). (B)

Percentage of cytokine-producing cells in dextramer®⁺ CD8⁺ T cells from R and NR. Statistical analysis was performed with the Mann-Whitney test. ns=not significant. (C) Correlation between cytokine-producing dextramer®⁺ CD8⁺ T cells and those expressing PD-1 (Spearman correlation analysis).

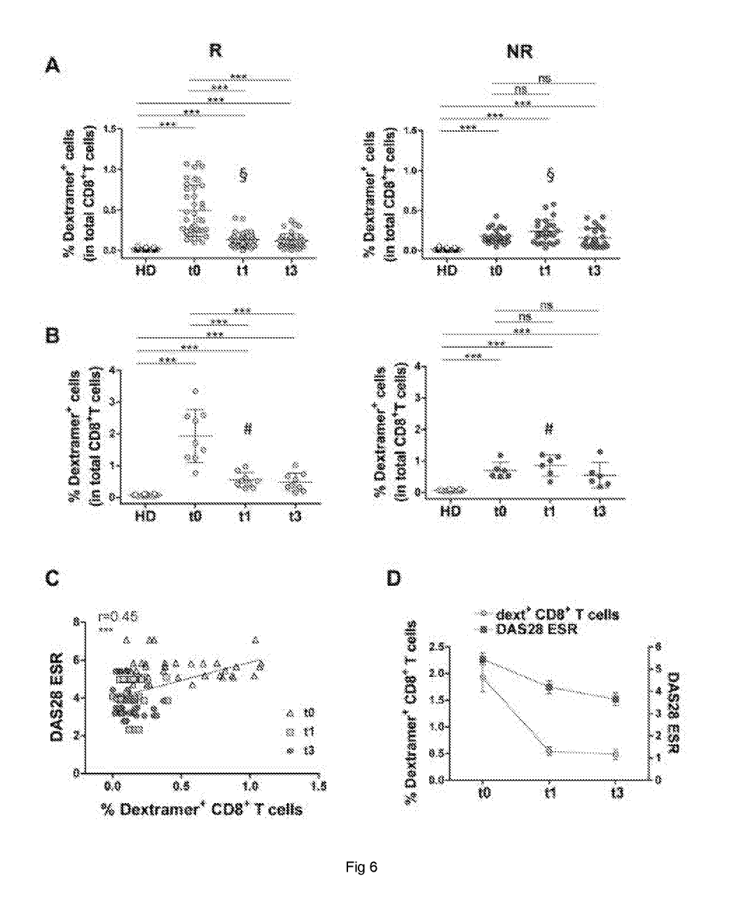

FIG. 6 Time course analysis performed longitudinally throughout the follow-up in TNF-α inhibitor-treated patients.

(A) Percentages of the single AE-specific Dextramer®⁺ CD8⁺ T cells determined before (TO) and after 1 (T1) and 3 (T3) months from the start of TNF-a inhibitor therapy in R and NR (each symbol represents a single AE-specific Dextramer®⁺ CD8⁺ T cell population). Statistical analysis was performed with Wilcoxon matched pairs test. ***p<0,0001; ns=not significant. The § symbol indicates the percentages of single AE-specific dextramer®⁺ CD8⁺ T cells that were compared between R and NR at the t1 (**p<0,001) (B) Sum of percentages of the single AE-specific dextramer®⁺ CD8⁺ T cells in single R and NR (each symbol represents a single patient). Statistical analysis was performed by Wilcoxon matched pairs test. ***p<0,0001; ns=not significant. The # symbol indicates the percentages of single AE-specific dextramer®⁺ CD8⁺ T cells that were compared between R and NR at the t1 (**p<0,001) (C) Correlation between DAS28ESR and the percentage of AE-specific dextramer®⁺ CD8⁺ T cells in R (Spearman correlation analysis). (D) Parallel follow-up of AE-specific dextramer®⁺ CD8⁺ T cell analyses and DAS28ESR at T0, T1, and T3 from the start of the TNF-α inhibitor therapy in R.

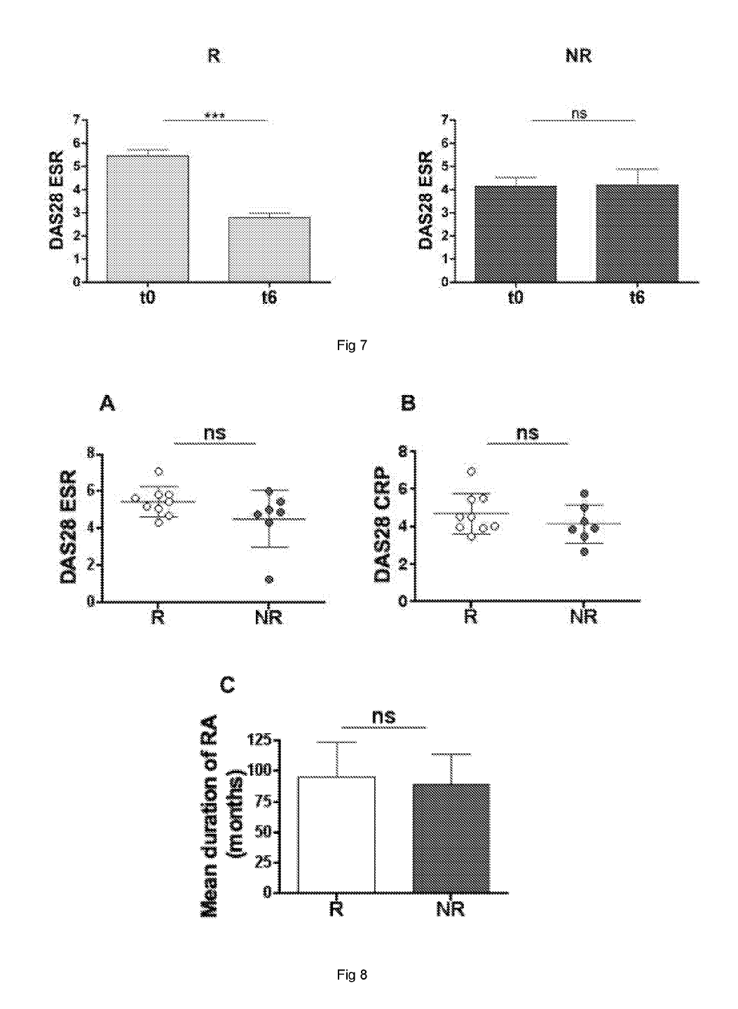

FIG. 7 (=suppl FIG. 1) DAS28 ESR score in R and NR.

Patients were assessed for overall disease activity using the DAS28, and categorized in R or NR according to the EULAR criteria 6 months after the start of treatment. An improvement of the DAS28 >0,6 was considered a response to therapy. Statistical analysis was performed with Paired t test. ***p<0,0001. ns=not significant.

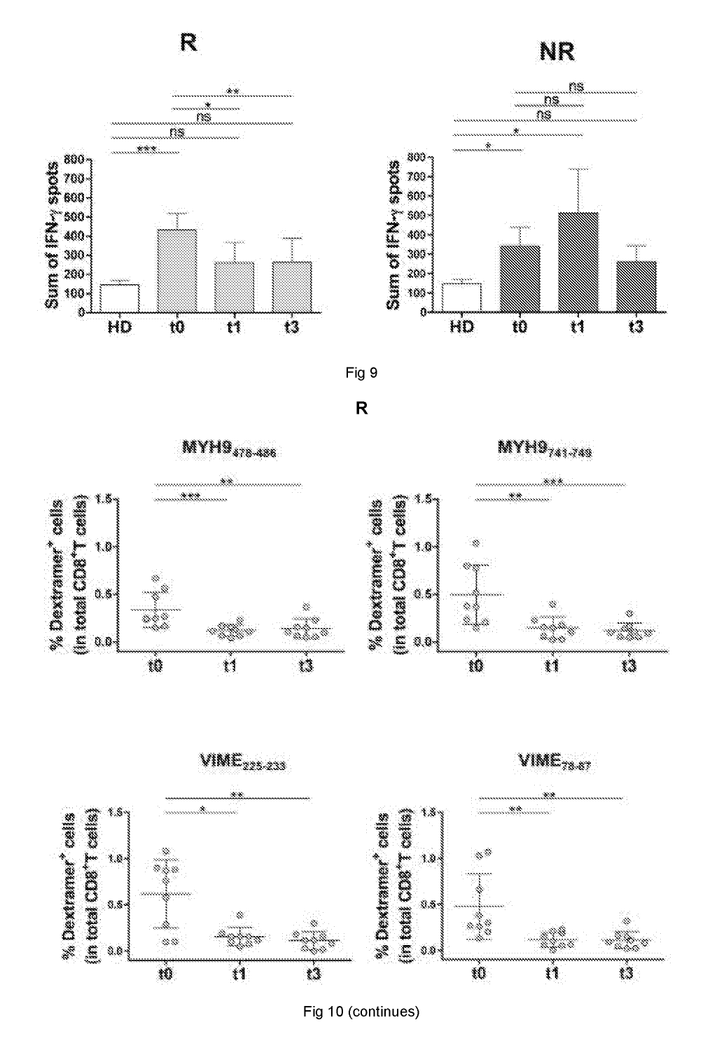

FIG. 8 (=suppl FIG. 2) Clinical criteria do not discriminate R and NR.

DAS28 ESR values (A), DAS28 CRP values (B), and RA duration (months) (C), before the start of TNF-α inhibitor therapy, in R and NR. Statistical analysis was performed with the Mann-Whitney test. ns=not significant.

FIG. 9 (=suppl FIG. 3) Follow-up of IFN-γ⁺ CD8⁺ T cells specific for AE as detected by ELISPOT.

Sum of IFN-γ⁺ cell spots formed in response to AE pools, analysed in R and NR at T0, T1, and T3 from the start of anti-TNF-α therapy. Statistical analysis was performed with Wilcoxon matched pairs test. *p<0,01; **p<0,001;

***p <0,0001. ns=not significant.

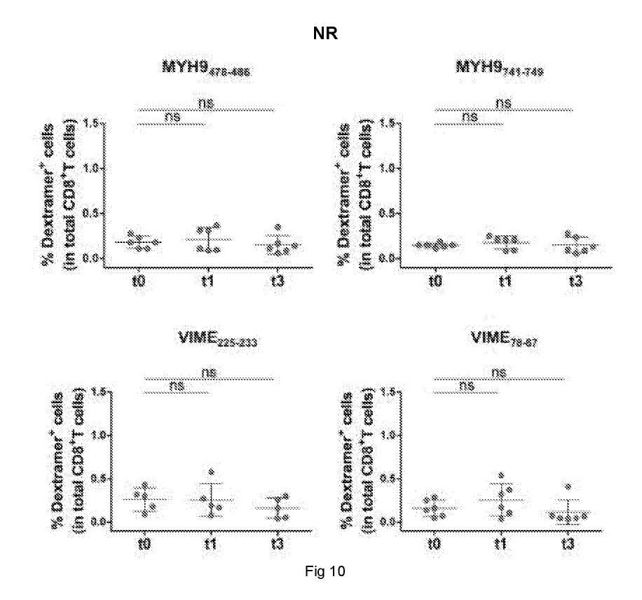

FIG. 10 (=suppl FIG. 4). Time course analyses of the Dextramer®⁺ CD8⁺ T cells, specific for the single AE indicated in R and NR. Analyses were performed at T0, T1, and T3 from the start of TNF-α inhibitor therapy. Statistical analysis was performed with Wilcoxon matched pairs test. *p<0,01; **p<0,001; ***p<0,0001. ns=not significant.

### DETAILED DESCRIPTION OF THE INVENTION

As explained in the summary above, the present invention stems from the discovery that, in autoimmune diseases, the proportion of circulating apoptotic lymphocytes is correlated with the frequency of CD8 T lymphocytes specific for the peptides (epitopes) derived from apoptotic antigens, (i.e., from antigenic fragments derived from apoptotic T cells that are processed and cross-presented by dendritic cells (DC) to an enormous repertoire of specific CD8 T lymphocytes), and that the frequency of these CD8 T lymphocytes specific to Apoptotic Epitopes is correlated with disease activity, disease progression, and is predictive of the response to certain therapies. As already mentioned, the frequencies of CD8 T lymphocytes specific against Apoptotic Epitopes are significantly higher in patients affected by autoimmune diseases than in control healthy subjects and are correlated with disease activity, resulting significantly higher in patients that will respond (responsive) to certain therapies than in non-responsive patients. Therefore, determining the frequency of CD8 T lymphocytes specific for apoptotic peptides (autoreactive) enables to have a biomarker predictive of response to therapy.

In the present description, in all embodiments described, Apoptotic Epitopes of human CD8⁺ T cells are to be understood as epitopes derived from cleavage by caspases (caspase cleavage) to one or more human cellular proteins, therefore producing fragments processed by dendritic cells (DC) which subsequently present a high proportion of distinct epitopes in these fragments (Apoptotic Epitopes, or AE) by the classic MHC (major histocompatibility complex) class 1 pathway to a wide repertoire of autoreactive CD8⁺ T cells.

In other terms, T cell epitopes, generated by caspase cleavage of cellular proteins, recognized by CD8⁺ T cells, are defined as Apoptotic Epitopes of CD8⁺ T cells. This definition is known in the literature and is, for instance, described in detail in Rawson et al, 2007. Examples of such epitopes are represented by peptides listed in Table 1 in the present description.

Therefore, the Apoptotic Epitopes according to the present invention are antigenic peptides derived from apoptotic T cells, and can be peptides derived from proteins of apoptotic T lymphocytes and can bind MCH class I molecules belonging to the same HLA haplotype or to different HLA haplotypes.

The term “Apoptotic Epitopes” may be replaced throughout the present description and in the claims by any one of the definitions provided above, or even by one of the following terms: “antigenic peptides derived from apoptotic T cells”, “antigenic peptides derived by apoptotic T cells generated from caspase cleavage of cellular proteins”, or “Apoptotic Epitopes according to Table 1”; in other terms, those are antigenic fragments derived from apoptotic T cells, that are processed and cross-presented by dendritic cells to an enormous repertoire of specific CD8 T lymphocytes.

Such peptides can be identified by the expert in the field by analysis of T cells proteome, as described, e.g., in Apoptotic cells overexpress vinculin and induce vinculin-specific cytotoxic T cell cross-priming. Nature Med. 7:807-813, 2001., and in Rawson et al, Cross-presentation of caspase-cleaved apoptotic self antigens in HIV infection. Nat. Med. 13: 1431-9, 2007. In the paper by Rawson et al, Nat Med 2007, the method of isolation of proteins derived by apoptotic T lymphocytes is described in detail. In particular, in the supplementary information available online, there are described: preparation of apoptotic lymphocytes, two-dimensional electrophoresis (2DE) of apoptotic T cells, protein isolation from 2DE gels, and MALDI-TOF-MS analysis .

Caspase-cleaved cellular proteins identified herein are, e.g.:
Actin cytoplasmic 1 (ACTB, reference sequence having accession number P02570);
heterogeneous nuclear riboprotein k (ROK, reference sequence having accession number Q07244);
lamin B1 (LAM1, reference sequence having accession number P20700)
non-muscle myosin heavy chain 9 (MYH9 reference sequence having accession number P35579)
vimentin (VIME reference sequence having accession number P08670)
proteasome component C2 (PSA1 reference sequence having accession number P25786)
rho GDP dissociation inhibitor 2 (GDIS reference sequence having accession number P52566)
60S acidic ribosomal protein C2 (RLA2 reference sequence having accession number P05387).

In the present description, in all embodiments, except where otherwise indicated, by “Apoptotic Epitopes bound by Human CD8⁺ T cells” short peptides of a length ranging from 8 and 12 amino acids are meant, capable of being bound by CD8 T lymphocytes specific for apoptotic peptides (autoreactive).

Such peptides may be short fragments (by caspase cleavage) of one or more of the above-listed proteins (ACTB, ROK, LAM1, MYH9, VIME, PSA1, GDIS, RLA2).

Concrete examples of such peptides are represented by sequences 1-90 of the sequence list attached to the present application and summarized in Table 1 below.

TABLE 1
HLA-A2 binding peptides derived
from apoptotic cell-associated proteins
detected by proteomic analyses.

| | PROTEIN | | |
|---|---|---|---|
| SEQ | (ACCESSION | Position | |
| ID | NUMBER) | of 1^{st} aa | SEQUENCE |
| 1 | ACTB (P02570) | 131 | AMYVAIQAV |
| 2 | Pool 1 | 319 | ALAPSTMKI |
| 3 | | 266 | FLGMESCGI |
| 4 | | 312 | RMQKEITAL |
| 5 | | 348 | SLSTFQQMWI |
| 6 | | 46 | GMGQKDSYV |
| 7 | ROK (Q07244) | 154 | SLAGGIIGV |
| 8 | Pool 2 | 67 | ALRTDYNASV |
| 9 | | 193 | VLIGGKPDRV |
| 10 | | 209 | ILDLISESPI |
| 11 | | 122 | QLPLESDAV |
| 12 | LAM1 (P20700) | 496 | TIWAANAGV |
| 13 | Pool 3 | 41 | RLAVYIDKV |
| 14 | | 301 | SLSSQLSNL |
| 15 | | 361 | QLLDVKLAL |
| 16 | | 291 | ELMESRMRI |
| 17 | | 355 | QLNDYEQLL |
| 18 | | 388 | KLSPSPSSRV |
| 19 | | 488 | VLKAGQTVTI |
| 20 | | 378 | KLLEGEEERL |
| 21 | MYH9 (P35579) | 9 | YLYVDKNFI |
| 22 | Pool 4 | 108 | GLIYTYSGL |
| 23 | | 111 | YTYSGLFCV |
| 24 | | 145 | EMPPHIYAI |
| 25 | | 186 | KVIQYLAYV |
| 26 | | 478 | QLFNKHTMFI |
| 27 | | 584 | WLMKNMDPL |
| 28 | | 653 | QLAKLMATL |
| 29 | | 111 | YTYSGLFCVV |
| 30 | MYH9 (P35579) | 424 | RMFRWLVLRI |
| 31 | Pool 5 | 478 | QLFMHTMFIL |
| 32 | | 302 | FLSNGHVTI |
| 33 | | 338 | GLLRVISGV |
| 34 | | 412 | FAIEALAKA |
| 35 | | 450 | ILDIAGFEI |
| 36 | | 733 | FMDGKQACV |
| 37 | | 741 | VLMIKALEL |
| 38 | | 1277 | KLQVELDNV |
| 39 | MYH9 (P35579) | 1843 | KLKDVLLQV |
| 40 | Pool 6 | 279 | YLLSGAGEHL |
| 41 | | 733 | FMDGKQACVL |
| 42 | | 1920 | KLRRGDLPFV |
| 43 | | 210 | QLLQANPIL |
| 44 | | 847 | MMAKEEELV |
| 45 | | 877 | QLMAEKLQL |
| 46 | | 1726 | RLEARIAQL |
| 47 | | 1793 | KLQEMEGTV |
| 48 | MYH9 (P35579) | 660 | TLRNTNPNFV |
| 49 | Pool 7 | 688 | VLDQLRCNGV |
| 50 | | 752 | NLYRIGQSKV |
| 51 | | 248 | YIVGANIET |
| 52 | | 1540 | QLEELEDEL |
| 53 | | 161 | MMQDREDQSI |
| 54 | | 821 | KLRNWQWWRL |
| 55 | | 846 | EMMAKEEELV |
| 56 | GDIS (P52566) | 100 | VLKEGSEYRV |
| 57 | Pool 8 | 186 | HLSWEWNLSI |
| 58 | | 37 | EMDKDDESL |
| 59 | | 51 | TLLGDGIVV |
| 60 | VIME (P08670) | 176 | NLAEDIMRL |
| 61 | Pool 9 | 50 | SLIASSPGGD |
| 62 | | 68 | RLRSSVPGV |
| 63 | | 129 | ILLAELEQL |
| 64 | | 225 | SLQEEIAFL |
| 65 | | 78 | LLQDSVDFSL |
| 66 | | 79 | LQDSVDFSL |
| 67 | | 419 | SLNLRETNL |
| 68 | | 122 | FLEQQNKILL |
| 69 | | 370 | NMKEEMARHL |
| 70 | PSA1 (P25786) | 179 | FMECNLNEL |
| 71 | Pool 10 | 183 | NLNELVKHGL |
| 72 | | 175 | HMSEFMECNL |
| 73 | | 63 | ILHVDNHIGI |
| 74 | | 37 | GLKSKTHAV |
| 75 | | 110 | SLIGSKTQI |
| 76 | | 179 | FMECNLNELV |
| 77 | | 48 | ALKRAQSEL |
| 78 | | 76 | GLTADARLL |
| 79 | PSA1 (P25786) | 102 | PLPVSRLVSL |
| 80 | Pool 11 | 204 | DLTTKNVSI |
| 81 | | 45 | ELNGKNIEDV |
| 82 | | 55 | ELAAHQKKI |
| 83 | | 186 | ELVKHGLRAL |
| 84 | | 37 | GLKSKTHAVL |
| 85 | | 191 | GLRALRETL |
| 86 | | 55 | ELAAHQKKIL |
| 87 | | 97 | FVFDRPLPV |
| 88 | RLA2 (P05387) | 3 | YVASYLLAA |
| 89 | Pool 12 | 26 | ILDSVGIEA |
| 90 | | 3 | YVASYLLAAL |

In the present invention such peptides are preferably associated with dextramers of MHC class I molecules by dextramers (dextramer®) technology, which can be costumed by Immudex, Copenhagen, Denmark, for each desired HLA haplotype, like e.g. HLA-A and its allelic forms, HLA-B and its allelic forms, or HLA-C and its allelic forms.

For the purposes of the present description, in any embodiment, the term “biological sample” indicates a sample containing CD8⁺ T cells (CD8⁺ T lymphocytes) and could be, e.g., a blood sample, or a PBMC (peripheral blood mononucleated cells) sample.

For the purposes of the present description, in any embodiment, the term dextramers (dextramer®) denotes a molecular complex comprised of multiple MHC class I molecules, each of which conjugated with fluorescein and complexed to the synthetic apoptotic peptides identified by the Inventors and described above.

As described on the Immunodex site, http://www.immudex.com/technology/dextramer-technology.aspx, MHC Dextramer™ reagents consist in a dextran polymer backbone carrying an optimized number of MHC and fluorochrome molecules. Dextramer reagents carry more MHC molecules and fluorochromes than conventional MHC multimers. This enhances their avidity for specific T cells and enhances their labeling intensity, thereby increasing the resolution and the signal/background ratio. The dextran polymer backbone stabilizes the shape of bound proteins, i.e. the MHC-peptide complexes and the fluorochromes, and are therefore highly stable reagents. The site also shows a clear diagram of dextramers, reported below:

In other terms, using the definition provided by Wikipedia reported herein, dextramers are reagents fluorescently labeled with FITC, PE or APC, and contain MHC molecules attached to a dextran backbone, which are used to detect antigen-specific T-cells in fluid cells and solid tissue samples using flow cytometry.

For the purposes of the present description, in any embodiment, the term “biological medicaments that block and/or inhibit TNF” (Tumour Necrosis Factor) includes biological medicaments that block/inhibit TNFα, like, e.g., adalimumab, certolizumab pegol, etanercept, golimumab, infliximab;

By “biological medicaments that block and/or inhibit cytokines or cytokine receptors” are meant biological medicaments that block/inhibit one or more cytokines or cytokine receptors, like, e.g., tocilizumab, anakinra; by “biological medicaments against activated B cells” are meant biological medicaments that inhibit the functions of B lymphocytes and deplete them, like, e.g., rituximab, consisting of an antibody that recognizes molecule CD20 on B lymphocytes and destroys them; and, by “biological medicaments that inhibit T cell co-stimulation” are meant biological medicaments that inhibit T lymphocytes, like e.g. abatacept, which is comprised of IgG1 Fc region fused with the extracellular domain of molecule CTLA-4, that by binding molecules CD80 and CD86 on T lymphocytes prevents the latter molecules from being co-stimulated by antigen-presenting cells (i.e., DCs) thereby becoming anergic.

The term “biological medicaments” in the present description has the meaning recognized in the state of the art, i.e. it denotes medicaments that mime or inhibit the effects of natural substances present in the body, but are produced in laboratory.

In one embodiment, the present invention relates to the use of one or more MHC class I molecules dextramers (Dextramers®) associated with peptides corresponding to Apoptotic Epitopes of human CD8⁺ T cells, for the predictive prognosis of responsiveness or non-responsiveness to certain therapeutic treatments, or even for monitoring the effectiveness of said therapeutic treatments, in patients affected by autoimmune diseases.

The therapeutic treatments according to the invention are treatments with biological medicaments that block and/or inhibit TNFα and/or biological medicaments that block and/or inhibit cytokines or cytokine receptors and/or biological medicaments against B cells and/or biological medicaments that inhibit T cell co-stimulation. Said predictive prognosis can be carried out with the methods described below, by determination of the frequencies or of the number of CD8⁺ T lymphocytes specific for dextramers (Dextramers®) of MHC class I molecules associated with peptides corresponding to Apoptotic Epitopes of human CD8⁺ T cells and comparing this number with that obtained from control patients with known responsivity or relative to a cutoff value reported below.

The monitoring of the effectiveness of the therapeutic treatments can be carried out in patients, in which there are determined, in time, the frequencies (or the number) of CD8⁺ T lymphocytes specific for dextramers (Dextramers®) of MHC class I molecules associated with peptides corresponding to Apoptotic Epitopes of human CD8⁺ T cells at different times before and during the treatment, wherein a decrease of said T lymphocytes indicates effectiveness of therapy.

For the purposes of the present invention, said peptides can be, for instance, one or more peptides derived from caspase cleavage of proteins ACTB, ROK, LAM1, MYH9, VIME, PSA1, GDIS, RLA2 as defined above.

In one embodiment, said peptides can be one or more peptides as indicated in Table 1 above, i.e. one or more peptides selected in the group of peptides having SEQ ID from 1 to 90.

According to one embodiment of the invention, said peptides are one or more peptides derived from caspase cleavage of proteins ACTB, MYH9 and VIME as defined above.

In a specific embodiment, said one or more peptides may be selected in the group of peptides having SEQ ID 3 (hereinafter also denoted as ACTB ₂₆₆₋₂₇₄) SEQ ID NO 31 (hereinafter also denoted as MYH9₄₇₈₋₄₈₆) SEQ ID NO 37 (hereinafter also denoted as MYH9₇₄₁₋₇₄₉) SEQ ID 64 (hereinafter also denoted as VIME ₂₂₅₋₂₃₃), SEQ ID NO 65 (hereinafter also denoted as VIME ₇₈₋₈₇).

In a further embodiment, said one or more peptides consist in the peptides having SEQ ID 3 (hereinafter also indicated as ACTB₂₆₆₋₂₇₄) SEQ ID NO 31 (hereinafter also indicated as MYH9₄₇₈₋₄₈₆) SEQ ID NO 37 (hereinafter also indicated as MYH9₇₄₁₋₇₄₉), SEQ ID 64 (hereinafter also indicated as VIME₂₂₅₋₂₃₃), SEQ ID NO 65 (hereinafter also indicated as VIME₇₈₋₈₇).

As mentioned above, for the purposes of the present invention, the peptides in any one of the forms indicated above can be used for the predictive prognosis of responsiveness to certain therapeutic treatments (as defined in the present description) and/or the monitoring of the effectiveness of said therapeutic treatments in patients affected by autoimmune diseases. Said autoimmune diseases could be, e.g., diseases selected in the group comprising Rheumatoid arthritis (RA), Systemic lupus erythematosus (SLE), Scleroderma, Crohn's disease, Ulcerative colitis, Dermatomyositis, Anti-phospholipid antibody syndrome, Burger's disease, Hashimoto's thyroiditis.

According to the present invention, said therapeutic treatments could be therapeutic treatments with one or more biological medicaments selected among biological medicaments that block and/or inhibit TNF and/or biological medicaments that block and/or inhibit cytokines or cytokine receptors and/or biological medicaments against B cells and/or biological medicaments that inhibit T cell co-stimulation.

Said biological medicaments that block and/or inhibit TNF may be, e.g., TNFα blockers/inhibitors selected in the group comprising adalimumab, certolizumab pegol, etanercept, golimumab, infliximab.

Said biological medicaments that block and/or inhibit cytokines or cytokine receptors are medicaments capable of blocking/inhibiting one or more cytokines or cytokine receptors, even indirecly. Said medicaments may be, e.g., blockers/inhibitors of any one of cytokines IL-1α, IL-1β, IL-17, IL-22, IL-18, IL-33 and IL-6 or of a receptor thereof and can be selected, e.g., in the group comprising, e.g., the blocker of IL-6 receptor (tocilizumab), the anti-IL-1 (anakinra).

Said biological medicaments against B cells are medicaments against activated B cells, i.e. biological medicaments that inhibit the functions of B lymphocytes and deplete them, like, e.g., rituximab, consisting of an antibody that recognizes the CD20 molecule on B lymphocytes and destroys them.

Said biological medicaments that inhibit T cell co-stimulation are medicaments like, e.g., abatacept, which is comprised of the IgG1 Fc region fused with the extracellular domain of the molecule CTLA-4, which by binding the molecules CD80 and CD86 on the T lymphocytes prevents the latter molecules from being co-stimulated by the antigen-presenting cells (i.e., DCs) thereby becoming anergic.

The responsiveness to said treatments or the effectiveness thereof, as emerges from what described above, can be ascertained by using the peptides and the methods described in the present description.

An ex vivo method for the predictive prognosis of responsiveness or non-responsiveness to treatments with biological medicaments that block and/or inhibit TNF, and/or biological medicaments that block and/or inhibit cytokines or cytokine receptors and/or biological medicaments against B cells and/or biological medicaments that inhibit T cell co-stimulation in patients affected by autoimmune diseases, comprising the following steps:
a. contacting a biological sample to be analysed comprising CD8⁺ T cells and, concomitantly, a control biological sample comprising CD8⁺ T cells representative of patients affected by autoimmune diseases that are responsive and/or representative of patients affected by autoimmune diseases that are non-responsive to said treatments, with one or more dextramers (Dextramers®) of MHC class I molecules associated with peptides corresponding to Apoptotic Epitopes of human CD8⁺ T cells;
b. quantifying the number of CD8⁺ T cells specifically binding said one or more dextramers in each sample;
c. comparing the amount of CD8⁺ T cells specifically binding said one or more dextramers in the analysed samples and predicting the responsiveness or non-responsiveness of the patient associated with said biological sample to be analysed to said treatments on the basis of the amount of CD8⁺ T cells specifically binding said one or more dextramers detected.

The term ex vivo, commonly used in scientific works, refers to testing, methods, measurements performed in or on tissues or cells outside of an organism, in an external environment, altering the natural conditions as little as possible. Ex vivo conditions enable testing on cells or tissues outside of the organism from which they originate. Therefore, the term ex vivo excludes in vivo embodiments. A main advantage of the use of ex vivo methods and techniques is the possibility of performing assays or measurements that otherwise, due to ethical or technical reasons, would not be possible on living organisms. For the purposes of the present invention, the term “sample to be analysed” refers to isolated samples, coming from patients affected by autoimmune diseases of whom the response to the above-indicated therapies is not known, and for whom the predictive method of the invention is to be applied before subjecting them to therapeutic treatments.

The amount of CD8⁺ T cells specifically binding Apoptotic Epitopes (i.e., the dextramers (Dextramers®) of MHC class I molecules associated with peptides corresponding to Apoptotic Epitopes of human CD8⁺ T cells according to the invention, also indicated herein as dextramer®⁺ CD8⁺ T cells) can be quantified by calculating the percentage of dextramer®⁺ CD8⁺ T cells relative to the total of CD8⁺ T cells.

This can be carried out by using markers specific for CD8⁺ T cells known in the state of the art and counting said cells by cytofluorimeter (e.g., by labeling cells with an anti-CD8 antibody).

When using a sample of responsive patients as control sample, if the sample to be analysed has a percentage of dextramer®⁺ CD8⁺ T cells, against the total of CD8⁺ T cells, comparable to that of the control, it will be a sample coming from a responsive patient (responder); if the percentage of dextramer®⁺ CD8⁺ T cells against the total of CD8⁺ T cells is instead significantly lower than that of the control, it will be a sample coming from a patient that will not respond effectively to the therapeutic treatment with biological medicaments as defined herein.

Conversely, when using a sample of non-responsive patients as control sample, if the sample to be analysed has a percentage of dextramer®⁺ CD8⁺ T cells against the total of CD8⁺ T cells comparable to that of the control, it will be a sample coming from a non-responsive patient; if instead the percentage of dextramer®⁺ CD8⁺ T cells against the total of CD8⁺ T cells in the sample to be analysed is instead significantly greater than that of the control, it will be a sample coming from a patient who will respond effectively to the therapeutic treatment with biological medicaments as defined herein. As mentioned above, the peptides of the present invention enable to quantify the number or the frequency of CD8⁺ T cells binding Apoptotic Epitopes; such quantifying enables to predict who will respond or not respond to the treatment before starting the same treatment. In particular, the data reported in the figures and in the examples below demonstrate that patients with frequencies of CD8⁺ T cells specific for Apoptotic Epitopes significantly higher than in the other patients will effectively respond to the therapeutic treatment as defined herein, whereas those with frequencies significantly lower than the former will not respond.

The percentage of CD8⁺ T cells specific for Apoptotic Epitopes conjugated with dextramers (in the present description also indicated as dextramer®⁺), and therefore of CD8⁺ T cells binding Apoptotic Epitopes, can be calculated by flow cytometry analysis. Therefore, according to an embodiment of the invention, in the above-indicated method there will be used a control biological sample representative of patients affected by autoimmune diseases that are responsive to said treatments, and the detection of a percent of CD8⁺ T cells specifically binding the dextramers Dextramers®) of MHC class I molecules associated with peptides corresponding to Apoptotic Epitopes of human CD8⁺ T cells in the sample to be analysed similar to the percent of CD8⁺ T cells specifically binding said dextramers in the control sample is predictive of responsiveness to said treatments, whereas the detection of a percent of CD8⁺ T cells specifically binding said dextramers in the sample to be analysed lower than the percent of CD8⁺ T cells specifically binding said dextramers in the control sample is predictive of non-responsiveness to said treatments.

When referring to the percentage of CD8⁺ T cells specifically binding dextramers (Dextramers®) of MHC class I molecules associated with peptides corresponding to Apoptotic Epitopes of human CD8⁺ T cells (i.e., Apoptotic Epitopes) in any embodiment of the present invention, it is meant the percentage of such cells against the total of CD8⁺ T cells analysed.

According to another embodiment, said control biological sample is representative of patients affected by autoimmune diseases not responsive to said treatments, and the detection of a percent of CD8⁺ T cells specifically binding dextramers (Dextramers®) of MHC class I molecules associated with peptides corresponding to Apoptotic Epitopes of human CD8⁺ T cells of the invention in the sample to be analysed higher than the percent of CD8⁺ T cells specifically binding said dextramers in the control sample is predictive of responsiveness to said treatments, whereas the detection of a percent of CD8⁺ T cells specifically binding said dextramers in the sample to be analysed similar to the percent of CD8⁺ T cells specifically binding said dextramers in the control sample is predictive of non-responsiveness to said treatments.

Of course, the method according to the invention can comprise a step a. which comprises contacting said one or more dextramers (Dextramers®) of MHC class I molecules associated with peptides corresponding to Apoptotic Epitopes of human CD8⁺ T cells with a biological sample to be analysed comprising CD8⁺ T cells and, concomitantly, a control biological sample comprising CD8⁺ T cells representative of patients affected by autoimmune diseases that are responsive and a control biological sample comprising CD8⁺ T cells representative of patients affected by autoimmune diseases, that are non-responsive to said treatments.

Moreover, the method can also comprise the use of a control sample representative of healthy individuals, the percentage of CD8⁺ T cells specifically binding Apoptotic Epitopes in this sample will be substantially 0.

The invention also provides an ex vivo method for the predictive prognosis of responsiveness or non-responsiveness to treatments with biological medicaments that block and/or inhibit TNFα and/or biological medicaments that block and/or inhibit cytokines or cytokine receptors and/or biological medicaments against B cells and/or biological medicaments that inhibit T cell co-stimulation in patients affected by autoimmune diseases, comprising the following steps:
a. contacting a biological sample to be analysed comprising CD8⁺ T cells with one or more p dextramers (Dextramers®) of MHC class I molecules associated with peptides corresponding to Apoptotic Epitopes of human CD8⁺ T cells;
b. quantifying the percent of CD8⁺ T cells specifically binding said one or more dextramers in the sample against the total of CD8⁺ T cells, wherein the presence of a percent of CD8⁺ T cells specifically binding said dextramers against the total number of CD8⁺ T cells ≧0.5% is predictive of responsiveness to said treatments.

Moreover, the invention also provides an ex vivo method for the predictive prognosis of responsiveness or non-responsiveness to treatments with biological medicaments that block and/or inhibit TNF, and/or biological medicaments that block and/or inhibit cytokines or cytokine receptors and/or biological medicaments against B cells and/or biological medicaments that inhibit T cell co-stimulation in patients affected by autoimmune diseases, comprising the following steps:
a. contacting a biological sample to be analysed, comprising CD8⁺ T cells, with one or more dextramers (Dextramers®) of MHC class I molecules associated with peptides corresponding to Apoptotic Epitopes of human CD8⁺ T cells;
b. quantifying the percent of CD8⁺ T cells specifically binding said one or more dextramers in the sample against the total of CD8⁺ T cells, wherein the presence of a percent of CD8⁺ T cells specifically binding said dextramers against the total number of CD8⁺ T cells ≧0.235% has a predictive value of a 78% responsiveness to said treatments whereas a percent <0.235% has a predictive value of a 75% non-responsiveness to said treatments.

The invention further provides an ex vivo method for monitoring the therapeutic effectiveness of treatments with biological medicaments that block and/or inhibit TNFα and/or biological medicaments that block and/or inhibit cytokines or cytokine receptors and/or biological medicaments against B cells and/or biological medicaments that inhibit T cell co-stimulation in a patient affected by autoimmune disease, comprising the following steps:
a. contacting biological samples comprising CD8⁺ T cells obtained in subsequent moments of time before and during said treatments with one or more dextramers (Dextramers®) of MHC class I molecules associated with peptides corresponding to Apoptotic Epitopes of human CD8⁺ T cells;
b. quantifying the percent of CD8⁺ T cells specifically binding said one or more dextramers in each sample against the total of CD8⁺ T cells;
c. evaluating the variation of said percent of CD8⁺ T cells specifically binding said one or more dextramers in said samples in time, wherein a decrease in the percentage of CD8⁺ T cells bound by said one or more dextramers in said samples in time indicates an effective therapy.

The Authors of the present invention in fact demonstrated, as reported in FIG. 6 and in the related examples, that in patients in which responsivity to the therapeutic treatments as described herein is foreseen by applying the predictive methods according to the invention, a decrease in time of the percentage of CD8⁺ T cells specifically binding Apoptotic Epitopes as defined herein (or peptides corresponding thereto) against the total of CD8⁺ T cells is observed.

All indications provided above with regard to dextramers (Dextramers®) of MHC class I molecules associated with peptides corresponding to Apoptotic Epitopes of human CD8⁺ T cells, to peptides, to therapeutic treatments and the suitable biological samples, apply to all of the above-described methods.

However, in short, the peptides in the one or more dextramers (Dextramers®) of MHC class I molecules associated with peptides corresponding to Apoptotic Epitopes of human CD8⁺ T cells in the above-described methods can be prepared as described above, or can be selected in the group of peptides having SEQ ID from 1 to 90.

In a specific embodiment, the peptides can be selected in the group comprised of SEQ ID 3, SEQ ID NO 31, SEQ ID NO 37, SEQ ID 64, SEQ ID NO 65.

The methods of the invention can be applied to patients affected by autoimmune diseases in general; specifically, the autoimmune diseases can be those selected in the group comprising Rheumatoid arthritis (RA), Systemic lupus erythematosus (SLE), Scleroderma, Crohn's disease, Ulcerative colitis, Dermatomyositis, Anti-phospholipid antibody syndrome, Burger's disease, Hashimoto's thyroiditis.

The treatments to which the methods of the invention refer include treatments with biological medicaments that block and/or inhibit TNFα, like, e.g., medicaments selected in the group comprising adalimumab, certolizumab pegol, etanercept, golimumab, infliximab; treatments with biological medicaments that block and/or inhibit cytokines or cytokine receptors, like, e.g., medicaments selected in the group comprising tocilizumab, anakinra; treatments with biological medicaments against B cells, like, e.g., rituximab; treatments with biological medicaments that inhibit T cell co-stimulation, like, e.g., abatacept.

Moreover, the invention relates to a kit for the predictive prognosis of the responsiveness to treatment of one or more diseases with biological medicaments that block and/or inhibit TNFα and/or biological medicaments that block and/or inhibit cytokines or cytokine receptors and/or biological medicaments against B cells and/or biological medicaments that inhibit T cell co-stimulation and/or for monitoring the effectiveness of said treatments with said medicaments in responsive patients, comprising

one or more MHC class I molecules dextramers (Dextramers®) associated with peptides corresponding to Apoptotic Epitopes of human CD8+ T cells in one or more aliquots,

one or more aliquots of a sample representative of individuals affected by an autoimmune disease that are responsive to said treatments and one or more aliquots of a sample representative of individuals affected by said autoimmune disease that are non-responsive to said treatments and, optionally, one or more aliquots of a control sample representative of healthy individuals.

The one or more aliquots of a sample representative of individuals affected by an autoimmune disease that are responsive to said treatments and one or more aliquots of a sample representative of individuals affected by said autoimmune disease that are non-responsive to said treatment and, optionally, one or more aliquots of a control sample representative of healthy individuals are provided as control in order to enable the implementation of one or more of the methods of the invention by the use of the kit.

The aliquots of samples representative of individuals affected by an autoimmune disease that are responsive, and of individuals that are non-responsive to the therapeutic treatments as defined herein, can be aliquots of samples representative of the two typologies of patients for patients affected by an autoimmune disease in general; in particular, said aliquots could be of patients affected by a disease selected in the group comprising Rheumatoid arthritis (RA), Systemic lupus erythematosus (SLE), Scleroderma, Crohn's disease, Ulcerative colitis, Dermatomyositis, Anti-phospholipid antibody syndrome, Burger's disease, Hashimoto's thyroiditis.

According to the invention, the kit could comprise pairs of control aliquots (responsive patients and non-responsive patients) for one or more of each of the above-listed diseases.

All indications given above with regard to the dextramers, peptides, therapeutic treatments and suitable biological samples apply also to the above-described kit. However, in short, the peptides in the dextramers (Dextramers®) of MHC class I molecules associated with peptides corresponding to Apoptotic Epitopes of human CD8⁺ T cells in the above-described methods can be prepared as described above, or can be selected in the group of peptides having SEQ ID from 1 to 90.

In a specific embodiment, the peptides can be selected in the group consisting of SEQ ID 3, SEQ ID NO 31, SEQ ID NO 37, SEQ ID 64, SEQ ID NO 65.

The treatments to which the kit of the invention relates include treatments with biological medicaments that block and/or inhibit TNFα, like, e.g., medicaments selected in the group comprising adalimumab, certolizumab pegol, etanercept, golimumab, infliximab; treatments with biological medicaments that block and/or inhibit cytokines or cytokine receptors, like, e.g., medicaments selected in the group comprising tocilizumab, anakinra; treatments with biological medicaments against B cells, like, e.g., rituximab; treatments with biological medicaments that inhibit T cell co-stimulation, like, e.g., abatacept.

In all of the above-indicated embodiments the term “comprising” can be replaced by the term “consisting in”.

The following examples aim at illustrating embodiments of the invention and technical data demonstrating the validity thereof.

### EXAMPLES

Patients and Methods

Study Population

16 selected HLA-A2+ biologic-naive patients (i.e., patients that had never previously been treated with biological medicaments) affected by selected RA (F/M=15/1; median age 53 years, range 36-69 years, mean disease duration 96.6 months, range 6-240 months), who had shown a satisfactory response to conventional DMARDs (disease-modifying antirheumatic drugs), including methotrexate (associated or not with other anti-inflammatory/immunosuppressive drugs) and submitted to a subsequent treatment with Etanercept (according to the 1987 ACR criteria) were included in the study (Table 2 below). Each patient was given a standard dose of 50 mg Etanercept per week subcutaneously, and followed for clinical parameters. Clinical response was set in the present study as an improvement of the 28-joint-count Disease Activity Score (DAS28)>0.6 after 6 months of therapy according to EULAR response criteria. Of the 16 patients, 9 resulted responders (R) and 7 non-responders (NR) (Table 2 and FIG. 7). No difference was shown between R and NR in terms of duration of disease, disease activity (as calculated by DAS28-ESR or DAS28-CRP), and serum levels of ACPA or RF before the therapy start (Table 2). 24 HLA-A2⁺ healthy donors (HDs) matched for sex and age with the patients were also included. The study protocol was approved by the relevant research ethics committee.

All patients and controls gave written informed consent.

Preliminary data on Systemic lupus erythematosus (SLE), Ulcerative colitis, Burger's disease and Hashimoto's thyroiditis show that the predictive method described herein can also be applied to those autoimmune diseases, supporting the applicability of the method to autoimmune diseases in general.

TABLE 2

| | | | Disease | | | | |
|---|---|---|---|---|---|---|---|
| | | | duration | | | DAS28 | Clinical |
| | Gender | Age | (months) | DMARDS* | ACPA** | ESR | result |
| 1 | F | 59 | 18 | MTX, HCQ | neg | 3.85 | Non-responder |
| 2 | F | 59 | 84 | MTX | pos | 6.01 | Non-responder |
| 3 | F | 54 | 72 | MTX, HCQ, SSZ | pos | 5.82 | Responder |
| 4 | F | 36 | 48 | MTX, HCQ | neg | 1.26 | Non-responder |
| 5 | F | 38 | 16 | MTX, HCQ | neg | 4.7 | Responder |
| 6 | F | 68 | 120 | MTX, HCQ | neg | 5.44 | Non-responder |
| 7 | M | 51 | 18 | MTX, HCQ, SSZ | pos | 5.08 | Responder |
| 8 | F | 43 | 60 | MTX | pos | 5.02 | Non-responder |
| 9 | F | 62 | 84 | MTX, LEF | neg | 4.91 | Non-responder |
| 10 | F | 69 | 84 | MTX | pos | 5.65 | Responder |
| 11 | F | 48 | 240 | MTX, HCQ | neg | 5.18 | Responder |
| 12 | F | 60 | 6 | MTX | neg | 7.08 | Responder |
| 13 | F | 53 | 48 | MTX | neg | 5.44 | Responder |
| 14 | F | 56 | 156 | MTX | pos | 5.84 | Responder |
| 15 | F | 66 | 216 | MTX, HCQ | neg | 4.74 | Non-responder |
| 16 | F | 44 | 216 | SO, CsA, MTX, | pos | 4.32 | Responder |
| | | | | HCQ | | | |

*Disease Modifying Anti-Rheumatic Drugs
**Anti-citrullinated protein autoantibodies

Sythetic Peptides

HLA-2 binding peptides (nonamers or decamers) reported in SEQ ID NO 1-90 were derived from caspase-cleaved fragment of proteins
Actin cytoplasmic 1 (ACTB, reference sequence having accession number P02570);
heterogeneous nuclear riboprotein (ROK, reference sequence having accession number Q07244);
lamin B1 (LAM1, reference sequence having accession number P20700)
non-muscle myosin heavy chain 9 (MYH9 reference sequence having accession number P35579)
vimentin (VIME reference sequence having accession number P08670)
proteasome component C2 (PSA1 reference sequence having accession number P25786)
rho GDP dissociation inhibitor 2 (GDIS reference sequence having accession number P52566)
60S acidic ribosomal protein C2 (RLA2 reference sequence having accession number P05387).

The peptides have the sequences SEQ ID from 1 to 90 and are reported in Table 1 above.

The selected peptides were associated with dextramers of MHC class I molecules, thereby obtaining dextramers (Dextramers®) of MHC class I molecules associated with peptides corresponding to Apoptotic Epitopes of human CD8⁺ T cells by technology performed as commercial service by Immudex, Copenhagen, Denmark.

Cell Preparations

Peripheral blood mononuclear cells (PBMCs) were isolated by density gradient. Spontaneous apoptosis of T cells from patients was determined by staining fresh PBMCs with fluorescein isothiocyanate (FITC)-labeled Annexin-V (Biolegend), propidium iodide (PI) (Biolegend) and allophycocyanin (APC)-labeled anti-CD3 monoclonal antibody (mAb) (Biolegend).

Enzyme-linked Immunospot (ELISPOT) Assay

PBMCs following stimulation with 12 independent pools of AE (see Table 1 above) were tested by enzyme-linked immunospot (ELISPOT) assay. Briefly, 96-well millimeter high-affinity plates (Millipore Corporation, Bedford, Mass.) were coated with 10 μg/ml of capture mAb against IFN-γ (BD Bioscience) at 4° C. overnight. The plates were blocked for 2 hours with blocking solution (PBS containing 2% bovine serum albumin [BSA]). A total of 1×10⁵ PBMCs were added to each well and stimulated for 18 hours (h) with peptides. Biotinylated anti-IFN-γ (BD Bioscience) diluted to 5 mg/ml in Blocking Solution as indicated by the manufacturer was added and incubated for 2 h in 5% CO₂ at 37° C. Plates were washed, incubated with alkaline phosphatase (AKP)-streptavidin (BD Bioscience) and developed with Sigmafast BCIP®/NBT (Sigma). The reaction was stopped by rinsing the plates with distilled water. Each well was then examined for positive signals (dots). The number of dots in each well was counted by an ELISPOT reader system (AELVIS reader system). IFN-γ-secreting cells were expressed as IFN-γ spots per each 1×10⁶ cells. The IFN-γ spot values were subtracted from the background, which was below 10 IFN-γ spots in 1×10⁶ cells for each test.

Monoclonal Antibody and Dextramer® Staining

PBMCs were incubated with APC-labeled-HLA-A*0201 dextramer® complexed respectively to MYH9₄₇₈₋₄₈₆ (QLFNHTMFI, SEQ ID NO 31), MYH9₇₄₁₋₇₄₉ (VLMIKALEL SEQ ID NO 37), VIME₇₈₋₈₇ (LLQDSVDFSL SEQ ID NO 65), VIME₂₂₅₋₂₃₃ (SLQEEIAFL SEQ ID NO 64) or ACTB₂₆₆₋₂₇₄ (FLGMESCGI SEQ ID NO 3) peptides (Immudex, Copenhagen, Denmark). The incubation was performed in FACS buffer (PBS containing 2% human AB serum) at room temperature for 10 min, followed by washing and further surface staining with FITC-labeled mAb to CD8 (eBioscience).

In order to distinguish the various CD8 cells, also phycoerythrin-cyanine (PeCy)7-labeled mAb to PD-1 (eBioscience), AlexaFluor700-labeled mAb to CD69, PECF594-labeled mAb to HLA-DR, and a cocktail of labeled-mAbs and -reagents (APC-Cy7-labeled mAbs to CD4, CD14, CD16, CD19, and CD56 [Biolegend]) and Fixable Viability Dye eFluor 780 [eBioscience]) (dump channel), for 20 min at 4° C., were used. Dextramer®⁺ cells were analysed within a CD8⁺ T cell gate, whereas CD69⁺, HLA-DR⁺, or 1 PD+cells within dextramer®⁺CD8⁺ cells, after exclusion of B cells, monocytes, natural killer T (NKT) cells, NK cells, cells CD4⁺ T cells (dump channel). Cells were acquired with LSRFortessa cytometer (Becton Dickinson) and analysed with FlowJo software version 7.5.5 (Tree star, Inc. San Carlos, Calif.).

Intracellular Cytokine Labeling

Cytokine production was analysed by intracellular staining assay (ICS). PBMCs were incubated with or without the relevant peptides (20 μg/ml) plus anti-CD28 mAb (4 μg/ml) (BD Biosciences) and Protein Transport Inhibitor Cocktail (Brefeldin A and Monensin) (eBioscience), or with Cell Stimulation Cocktail as positive control (PMA, ionomycin, brefeldin A and monensin) (eBioscience), for 18 h at 37° C. Cells were washed, and stained with APC-labeled-HLA-A*0201 dextramers® complexed to the above-indicated peptides, PeCy7-labeled mAb to CD8 (Biolegend) and the dump channel reagents. Cells were fixed and permeabilised using Cytofix/Cytoperm solution (BD Biosciences) at 4° C. for 20 min, re-washed with Perm Wash Buffer (BD Biosciences), and stained with different combinations of AlexaFluor700-labeled IL17A (Biolegend), fluorescein-conjugated anti-IFN-γ (Biolegend) for 20 min at 4° C. Cells were washed, acquired with LSRFortessa cytometer (Becton Dickinson) and analysed with FlowJo software. IL-17, IFN-γ or IL-17/IFN-γ producing cells were analysed in CD8⁺ dextramer®⁺ T cells after exclusion of B cells, monocytes, NKT cells, NK cells, CD4⁺ T cells (dump channel).

Statistical Analyses

The collected data underwent statistical analysis by GraphPad Prism version 4 software (GraphPad Software). The data of comparison between healthy donors and patients, comparison in patients at different times, correlation between different tests performed and correlation between tests and clinical data were analysed, respectively, with Mann-Whitney test, Wilcoxon matched pairs test, linear regression and Spearman's correlation. The significance threshold was set at p=0.05. Receiver Operating Characteristic (ROC) analysis was performed to assess the predictive power of AE-specific CD8⁺ T cells. The area under the ROC curve (AUC) was calculated along with 95% confidence intervals (CI). In the study reported herein, AUC value indicates the ability of Dextramer®⁺ T cells to distinguish R and NR.

Results

Multispecific CD8⁺ T cell Responses to Apoptotic Epitopes (AE)

Initially, the Inventors analysed longitudinally the effector responses by the capacity of freshly isolated CD8⁺ T cells from either 12 of the 16 HLA-A2⁺ patients or 24 HDs, to form IFN-γ spots (in an ELISPOT assay) within 4 to 6 hours (h) of contact with 12 pools containing a total of 90 synthetic HLA-A2-binding apoptotic peptides (Table 1). Therefore, the Inventors defined these CD8⁺ T cells as “T_{EM}”, on the basis of their capacity to perform their effector functions promptly within few hours of antigenic stimulus. Each peptide pool was tested in triplicate. The synthetic peptides used were prepared according to the sequence of caspase-cleaved proteins that had been previously identified by the proteomic analyses of apoptotic T cells (e.g., fragments of ACTB, ROK, LAM1, MYH9, GDIS, VIME, PSA1, e RLA2 proteins as above-defined and described). From the study it emerged that the responses to AE by IFN-γ⁺ CD8⁺ T_{Em} cells were significantly higher and wider in the patients' pool than in the HD pool (FIG. 1). In particular, both the median number of IFN-γ spots formed by CD8⁺ T_{Em} cells from all patients with rheumatoid arthritis (RA patients) or HDs in response to the single peptide pool (responsiveness) (FIG. 1A), and the sum of IFN-γ spots formed in response to the total peptide repertoire by a single patient or HD (magnitude) (FIG. 1B) were significantly higher in RA patients than in HDs. The HLA-restriction of these responses was demonstrated both by blocking responses with an appropriate anti-class I mAb and by determining that no response was observed in HLA-AZ patients (data not shown). With this type of assay, no correlation was found between the ELISPOT responses to AE and the disease activity, as calculated by both DAS28-ESR and DAS28-CRP (data not shown). In addition, no difference in the AE repertoire recognized by IFN-γ⁺ CD8⁺ T_{EM} cells was observed between R and NR at the time point tested before the start of therapy (time 0) (FIG. 10).

AE-specific CD8⁺ T Cells as Detected by Dextramers® Peptides are Predictive of the Effect of TNF-α Inhibitor Therapy

To explore the possibility that the frequencies of CD8⁺ T cells specific to AE, as detected by ELISPOT assay, were not different between R and NR (FIG. 1A), because the technique used only identified IFN-γ⁺ cells, a class I molecule multimer technology was used which would allow to count the entire AE-specific CD8⁺ T cell population with the same epitope specificity, irrespective of their differentiation phase, as well as T cells with an “exhaustion phenotype”, representing the reducing capacity of cells to perform effector functions. AE-specific CD8⁺ T cells in the peripheral blood of 15 HLA-A2⁺ RA patients (of which 9 would be resulted R, and 6 NR) were enumerated, by using dextramers® of HLA-A*0201 molecules complexed with ACT B₂₆₆₋₂₇₄, MYH9₄₇₈₋₄₈₆, MYH9₇₄₁₋₇₄₉, VIME₇₈₋₈₇, or VIME₂₂₅₋₂₃₃ peptides (respectively, SEQ ID NO 3, 37, 31, 65, 64) (FIG. 2). Control dextramers® complexed to a non-natural irrelevant peptide were unable to stain CD8⁺ T cells in all samples analysed (data not shown). All patients presented frequencies of peripheral dextramer®⁺ CD8⁺ T cells significantly higher than HD, in terms both of responsiveness and magnitude (FIG. 2A-C). Amazingly, the total frequencies of AE-specific CD8⁺ T cells, as detected by dextramers®, were significantly higher in R patients than in NR patients at the time point tested before the start of therapy (time 0) (FIG. 2D,E). ROC analysis was performed to evaluate the discriminatory accuracy of AE-specific CD8⁺ T cells. When comparison was made between R and NR, the area under ROC curve (AUC) was 0.82 (95% CI=0.722-0.925, P<0.0001). By contrast, no difference in DAS28-ESR, DAS28-CRP, age, disease duration, or presence of ACPA between R and NR was observed at the time 0 (Suppl. Table 2 above and FIG. 8). The percentage of early apoptotic T cells (as detected by Annexin V staining) circulating in PBMCs was significantly more elevated both in the total patients than in HD (FIG. 3A, B), and (even more important) in R patients significantly more than in NR patients (FIG. 3C.): notably, it directly correlated with the frequency of AE-specific CD8⁺ T cells (FIG. 3D, E.), suggesting a possible cause-effect between the two events. A significant proportion of these AE-specific CD8⁺ T cells expressed late (e.g., HLA-DR and PD-1) activation markers, indicating that they are experienced T cells (FIG. 4A, B). However, in contrast to the total AE-specific CD8⁺ T cells that were significantly higher in R (see FIG. 2), the experienced

AE-specific CD8⁺ T cells (and in particular those expressing the PD-1 exhaustion marker) were more represented in NR than in R (FIG. 4A, B).

Importantly, PD-1 expression inversely correlated with the frequencies of the total AE-specific CD8⁺ T cells (FIG. 4C), a data suggesting a role of the inhibitory PD-1 molecule in tempering T cell survival/expansion, particularly in NR patients showing frequencies of these cells significantly lower than in R patients at the time 0 (see FIG. 2). To validate the antigen-specificity of AE-specific (dextramer®) CD8⁺ T cells, the Inventors analysed their capacity to produce inflammatory cytokines (IFN-γ IL-17) within a few h of contact with the relevant peptides and optimal concentrations of anti-CD28 mAb, which served as a surrogate costimulatory signal. Undetectable cytokine production was observed when AE-specific CD8⁺ dextramer®⁺ T cells of 20 HLA-A2⁺ HDs were stimulated with this procedure (data not shown). Notably, AE-specific CD8⁺ dextramer®⁺ T cells produced moderate amounts of IFN-γ or IL-17 in response to the relevant epitopes ex vivo, supporting that they were effectively antigen-specific T_{EM} cells (FIG. 5). These responses tended to be higher in the pool of R patients than in that of NR patients at the time 0 (FIG. 5A, B) (although not significantly), and were inversely correlated with the percentage of AE-specific CD8⁺ T cells expressing PD-1 (FIG. 5C).

Follow-Up of AE-Specific CD8⁺ T Cells in Rheumatoid Arthritis (RA) Patients Treated with TNF-α Inhibitors

Time course analyses of the disease, performed longitudinally throughout the follow-up in patients treated with TNF-α inhibitors, revealed, first, a significant decline of ELISPOT responses (that were equally represented in R and NR at the time 0 [see FIG. 1]) only in pool R (FIG. 9), despite of it not correlating with the decline of disease activity (data not shown). This, together with the finding that the IFN-γ⁺ ELISPOT assay did not display any predictive value (no difference was shown between R and NR at the time 0 [see FIG. 1]), led the Authors of the present invention to monitor frequencies of all the AE-specific CD8⁺ T cell populations with the same epitope specificity, irrespective of their differentiation stage (dextramer®⁺ cells), to verify if they may provide sensitive information during the time course analyses of the disease. The frequencies of AE-specific (dextramer®⁺) CD8⁺ T cells (capable of predicting the response to TNF-α inhibitor therapy [see FIG. 2]), dropped in a significant fashion since the first month of therapy in pool R, but not in pool NR (FIG. 6A, B), and in a manner considerably more sensitive than what observed by ELISPOT assay. It should be noted that the AE-specific CD8⁺ T cell frequency in pool R after 1 month of therapy decreased at a level significantly lower than the corresponding frequencies in pool NR (FIG. 6A, B). Importantly, the decrease of the AE-specific CD8⁺ T cells in R, but not in NR (not shown), was related with the reduction of clinical parameters (e.g., DARS-ESR) (FIG. 6C,D), strongly suggesting a relationship between these cells and the immunopathology and chronic evolution of RA. Notably, the difference between R and NR was confirmed also at the level of the frequency of CD8⁺ T cells specific to a single peptide (FIG. 10).

Discussion

The Inventors therefore demonstrated for the first time that frequency, multispecificity, and magnitude of CD8⁺ T cells directed to AE were significantly higher in RA patients as compared with HDs pool, and correlated with the disease activity, indicating, without wishing to be bound by theory, that they might contribute to its progression. Amazingly, the frequencies of AE-specific CD8⁺ T cells (as detected by dextramers® technology) were significantly higher in pool R than in pool NR at the time 0 (before the start of therapy), as indicated by the significant ROC sensitivity and specificity. This, together with the finding that no clinical criteria (including DAS28-ESR, DAS28-CRP, or ACPA) was capable to discriminate R from NR in many autoimmune diseases, suggest that the frequency of AE-specific CD8⁺ T cells represents a unique biomarker predicting the response to therapy with TNF-α inhibitors and other biological medicaments such as those listed in this description in patients with autoimmune diseases.

### SEQUENCE DESCRIPTION

The sequences are described in Table 1 above.

### REFERENCE

Franceschini et al, Plos Pathog 2012 8 (6) <<Polyfunctional Type-1, -2 and -17 CD8⁺ T Cell responses to Apoptotic Self-antigens Correlate with the chronic evolution with Hepatitis C Virus infection>>.

Propato et al, “Apoptotic cells overexpress vinculin and induce vinculin-specific cytotoxic T cell cross-priming”. Nature Med. 7:807-813, 2001.

WO2012/159993 “Method to prognose viral infections by measuring T cell responses or autoantibodies to apoptotic epitopes”

Rawson et al, “Cross-presentation of caspase-cleaved apoptotic self antigens in HIV infection”. Nat. Med. 13: 1431-9, 2007

1. Use of one or more MHC class I molecules dextramers) (Dextranners®) associated with peptides corresponding to Apoptotic Epitopes of human CD8+ T cells for the predictive prognosis of responsiveness or non-responsiveness to treatments and/or for monitoring the therapeutic effectiveness of treatments with biological medicaments that block and/or inhibit TNFα and/or biological medicaments that block and/or inhibit cytokines or cytokine receptors, and/or biological medicaments against B cells, and/or biological medicaments that inhibit T cell co-stimulation in patients affected by autoimmune diseases.

2. The use according to claim 1, wherein said one or more peptides corresponding to Apoptotic Epitopes of human CD8+ T cells are selected from the group consisting of peptides having SEQ ID NOS from 1 to 90.

3. The use according to claim 2 wherein said one or more peptides corresponding to Apoptotic Epitopes of human CD8+ T cells are selected from the group consisting of peptides having SEQ ID NO 3, SEQ ID NO 31, SEQ ID NO 37, SEQ ID NO 64, and SEQ ID NO 65.

4. The use according to claim 1, wherein said autoimmune diseases are selected from the group consisting of Rheumatoid arthritis (RA), Systemic lupus erythematosus (SLE), scleroderma, Crohn's disease, ulcerative colitis, dermatomyositis, Anti-phospholipid antibody syndrome, Burger's disease, and Hashimoto's thyroiditis.

5. The use according to claim 1 wherein said biological medicaments that block and/or inhibit TNFα are selected from the group consisting of adalimumab, certolizumab pegol, etanercept, golimumab, and infliximab.

6. The use according to claim 1 wherein said biological medicaments that block and/or inhibit cytokines or cytokine receptors are selected from the group consisting of tocilizumab and anakinra.

7. The use according to claim 1 wherein said biological medicaments against B cells are selected from the group consisting of rituximab.

8. The use according to claim 1 wherein said biological medicaments that inhibit T cell co-stimulation are selected from the group consisting of abatacept.

9. The use according to claim 1 wherein said monitoring is carried out on patients that are responsive to said treatments.

10. An ex vivo method for the predictive prognosis of responsiveness or non-responsiveness to treatments with biological medicaments that block and/or inhibit TNF, and/or biological medicaments that block and/or inhibit cytokines or cytokine receptors, and/or biological medicaments against B cells, and/or biological medicaments that inhibit T cell co-stimulation in patients affected by autoimmune diseases, comprising the following steps:
a. contacting a biological sample to be analysed, comprising CD8+ T cells and a control biological sample comprising CD8+ T cells representative of patients affected by autoimmune diseases that are responsive or non-responsive to said treatments, with one or more dextramers (Dextranners®)of MHC class I molecules associated with peptides corresponding to Apoptotic Epitopes of human CD8+ T cells;
b. quantifying the percent of CD8+ T cells specifically binding said one or more dextramers in each sample against the total of CD8+ T cells;
c. comparing the percent of CD8+ T cells specifically binding said one or more dextramers in the analysed samples and predicting the responsiveness or non-responsiveness to said treatments of the patient associated with said biological sample to be analysed on the basis of the percentage of CD8+ T cells specifically binding said one or more dextramers detected.

11. An ex vivo method according to claim 10 wherein said control biological sample is representative of patients affected by autoimmune diseases that are responsive to said treatments and wherein the detection of a percent of CD8+ T cells specifically binding said dextramers in the sample to be analysed similar to the percent of CD8+ T cells specifically binding said dextramers in the control sample is predictive of responsiveness to said treatments, whereas the detection of a percent of CD8+ T cells specifically binding said dextramers in the sample to be analysed lower than the percent of CD8+ T cells specifically binding said dextramers in the control sample is predictive of non-responsiveness to said treatments.

12. An ex vivo method according to claim 10 wherein said control biological sample is representative of patients affected by autoimmune diseases that are not responsive to said treatments and wherein the detection of a percent of CD8+ T cells specifically binding said dextramers in the sample to be analysed higher than the percent of CD8+ T cells specifically binding said dextramers in the control sample is predictive of responsiveness to said treatments, whereas the detection of a percent of CD8+ T cells specifically binding said dextramers in the sample to be analysed similar to the percent of CD8+ T cells specifically binding said dextramers in the control sample is predictive of non-responsiveness to said treatments.

13. An ex vivo method according to claim 10 wherein said step a. comprises contacting said one or more dextramers with a biological sample to be analysed comprising CD8+ T cells and, concomitantly, a control biological sample comprising CD8⁺ T cells representative of patients affected by autoimmune diseases that are responsive to said treatments and a control biological sample comprising CD8+ T cells representative of patients affected by autoimmune diseases that are non-responsive to said treatments.

14. An ex vivo method for the predictive prognosis of responsiveness or non-responsiveness to treatments with biological medicaments that block and/or inhibit TNF, and/or biological medicaments that block and/or inhibit cytokines or cytokine receptors, and/or biological medicaments against B cells, and/or biological medicaments that inhibit T cell co-stimulation in patients affected by autoimmune diseases, comprising the following steps:
a. contacting a biological sample to be analysed comprising CD8+ T cells with one or more dextramers (Dextranners®) of MHC class I molecules associated with peptides corresponding to Apoptotic Epitopes of Human CD8+ T cells;
b. quantifying the percent of CD8+ T cells specifically binding said one or more dextramers in the sample against the total of CD8+ T cells, wherein the presence of a percent of CD8+ T cells specifically binding said dextramers against the total number of CD8+ T cells ≧0.5% is predictive of responsiveness to said treatments.

15. An ex vivo method for the predictive prognosis of responsiveness or non-responsiveness to treatments with biological medicaments that block and/or inhibit TNF, and/or biological medicaments that block and/or inhibit cytokines or cytokine receptors and/or biological medicaments against B cells and/or biological medicaments that inhibit T cell co-stimulation in patients affected by autoimmune diseases, comprising the following steps:
a. contacting a biological sample to be analysed comprising CD8+ T cells with one or more dextramers (Dextranners®) of MHC class I molecules associated with peptides corresponding to Apoptotic Epitopes of human CD8+ T cells
b. quantifying the percent of CD8+ T cells specifically binding said one or more dextramers in the sample against the total of CD8+ T cells, wherein the presence of a percent of CD8+ T cells specifically binding said dextramers against the total number of CD8+ T cells ≧0.235% has a predictive value of a 78% responsiveness to said treatments whereas a percent <0.235% has a predictive value of a 75% non-responsiveness to said treatments.

16. An ex vivo method for monitoring the therapeutic effectiveness of treatments with biological medicaments that block and/or inhibit TNFα and/or biological medicaments that block and/or inhibit cytokines or cytokine receptors and/or biological medicaments against B cells and/or biological medicaments that inhibit T cell co-stimulation in a patient affected by an autoimmune disease, comprising the following steps:
a. contacting biological samples comprising CD8+ T cells obtained in subsequent moments of time before and during said treatments with one or more dextramers (Dextranners®) of MHC class I molecules associated with peptides corresponding to Apoptotic Epitopes of human CD8+ T cells
b. quantifying the percent of CD8+ T cells specifically binding said one or more dextramers in each sample against the total of CD8+ T cells
c. evaluating the variation of said percent of CD8+ T cells specifically binding said one or more dextramers in said samples in time,
wherein a decrease in the percent of CD8+ T cells bound by said one or more dextramers in said samples in time indicates an effective therapy.

17. A method according to claim 10 wherein said one or more peptides corresponding to Apoptotic Epitopes of human CD8+ T cells are selected from the group consisting of peptides having SEQ ID NOS from 1 to 90.

18. A method according to claim 17 wherein said one or more peptides corresponding to Apoptotic Epitopes of human CD8+ T cells are selected from the group consisting of peptides having SEQ ID NO 3, SEQ ID NO 31, SEQ ID NO 37, SEQ ID NO 64, and SEQ ID NO 65.

19. The method according to claim 10 wherein said autoimmune diseases are selected from the group consisting of Rheumatoid arthritis (RA), Systemic lupus erythematosus (SLE), Scleroderma, Crohn's disease, ulcerative colitis, dermatomyositis, anti-phospholipid antibody syndrome, Burger's disease, DEL Hashimoto's thyroiditis.

20. The method according to claim 10 wherein said biological medicaments that block and/or inhibit TNFα are selected from the group consisting of adalimumab, certolizumab pegol, etanercept, golimumab, and infliximab.

21. The method according to claim 10 wherein said biological medicaments that block and/or inhibit cytokines or cytokine receptors are selected from the group consisting of tocilizumab, and anakinra.

22. The method according to claim 10 wherein said biological medicaments against B cells are selected from the group consisting of rituximab.

23. The method according to claim 10 wherein said biological medicaments that inhibit T cell co-stimulation are selected from the group consisting of abatacept.

24. A kit for the predictive prognosis of the responsiveness to treatment of one or more diseases with biological medicaments that block and/or inhibit TNFα, and/or biological medicaments that block and/or inhibit cytokines or cytokine receptors and/or biological medicaments against B cells and/or biological medicaments that inhibit cell T co-stimulation and/or for monitoring the effectiveness of said treatment with said medicaments in responsive patients, comprising
one or more MHC class I molecules dextramers (Dextranners®) associated with peptides corresponding to Apoptotic Epitopes of human CD8+ T cells in one or more aliquots,
one or more aliquots of a control sample comprising human CD8+ T cells representative of healthy individuals, one or more aliquots of a sample comprising human CD8+ T cells representative of individuals affected by an autoimmune disease that are responsive to said treatments and one or more aliquots of a sample comprising human CD8+ T cells representative of individuals affected by said autoimmune disease that are non-responsive to said treatments.

25. The kit according to claim 24 wherein said one or more peptides corresponding to Apoptotic Epitopes of human CD8+ T cells are selected from the group consisting of peptides having SEQ ID NOS from 1 to 90.

26. The kit according to claim 25 wherein said one or more peptides corresponding to Apoptotic Epitopes of human CD8⁺ T cells are selected from the group consisting of peptides having SEQ ID NO 3, SEQ ID NO 31, SEQ ID NO 37, SEQ ID NO 64, and SEQ ID NO 65.

27. The kit according to claim 24 wherein autoimmune diseases are selected from the group consisting of Rheumatoid arthritis (RA), Systemic lupus erythematosus (SLE), scleroderma, Crohn's disease, ulcerative colitis, dermatomyositis, anti-phospholipid antibody syndrome, Burger's disease, and Hashimoto's thyroiditis.

28. The kit according to claim 24 wherein said biological medicaments that block and/or inhibit TNFα are selected from the group consisting of adalimumab, certolizumab pegol, etanercept, golimumab, and infliximab.

29. The kit according to claim 24 wherein said biological medicaments that block and/or inhibit cytokines or cytokine receptors are selected from the group consisting of tocilizumab and anakinra.

30. The kit according to claim 24 wherein said biological medicaments against B cells are selected from the group consisting of rituximab.

31. The kit according to claim 24 wherein said biological medicaments that inhibit T cell co-stimulation are selected from the group consisting of abatacept.


SEQUENCE LISTING (90 sequences)

SEQ ID NO: 1 - protein, 9 aa, Homo sapiens
AMYVAIQAV

SEQ ID NO: 2 - protein, 9 aa, Homo sapiens
ALAPSTMKI

SEQ ID NO: 3 - protein, 9 aa, Homo sapiens
FLGMESCGI

SEQ ID NO: 4 - protein, 9 aa, Homo sapiens
RMQKEITAL

SEQ ID NO: 5 - protein, 10 aa, Homo sapiens
SLSTFQQMWI

SEQ ID NO: 6 - protein, 9 aa, Homo sapiens
GMGQKDSYV

SEQ ID NO: 7 - protein, 9 aa, Homo sapiens
SLAGGIIGV

SEQ ID NO: 8 - protein, 10 aa, Homo sapiens
ALRTDYNASV

SEQ ID NO: 9 - protein, 10 aa, Homo sapiens
VLIGGKPDRV

SEQ ID NO: 10 - protein, 10 aa, Homo sapiens
ILDLISESPI

SEQ ID NO: 11 - protein, 9 aa, Homo sapiens
QLPLESDAV

SEQ ID NO: 12 - protein, 9 aa, Homo sapiens
TIWAANAGV

SEQ ID NO: 13 - protein, 9 aa, Homo sapiens
RLAVYIDKV

SEQ ID NO: 14 - protein, 9 aa, Homo sapiens
SLSSQLSNL

SEQ ID NO: 15 - protein, 9 aa, Homo sapiens
QLLDVKLAL

SEQ ID NO: 16 - protein, 9 aa, Homo sapiens
ELMESRMRI

SEQ ID NO: 17 - protein, 9 aa, Homo sapiens
QLNDYEQLL

SEQ ID NO: 18 - protein, 10 aa, Homo sapiens
KLSPSPSSRV

SEQ ID NO: 19 - protein, 10 aa, Homo sapiens
VLKAGQTVTI

SEQ ID NO: 20 - protein, 10 aa, Homo sapiens
KLLEGEEERL

SEQ ID NO: 21 - protein, 9 aa, Homo sapiens
YLYVDKNFI

SEQ ID NO: 22 - protein, 9 aa, Homo sapiens
GLIYTYSGL

SEQ ID NO: 23 - protein, 9 aa, Homo sapiens
YTYSGLFCV

SEQ ID NO: 24 - protein, 9 aa, Homo sapiens
EMPPHIYAI

SEQ ID NO: 25 - protein, 9 aa, Homo sapiens
KVIQYLAYV

SEQ ID NO: 26 - protein, 9 aa, Homo sapiens
QLFNHTMFI

SEQ ID NO: 27 - protein, 9 aa, Homo sapiens
WLMKNMDPL

SEQ ID NO: 28 - protein, 9 aa, Homo sapiens
QLAKLMATL

SEQ ID NO: 29 - protein, 10 aa, Homo sapiens
YTYSGLFCVV

SEQ ID NO: 30 - protein, 10 aa, Homo sapiens
RMFRWLVLRI

SEQ ID NO: 31 - protein, 10 aa, Homo sapiens
QLFNHTMFIL

SEQ ID NO: 32 - protein, 9 aa, Homo sapiens
FLSNGHVTI

SEQ ID NO: 33 - protein, 9 aa, Homo sapiens
GLLRVISGV

SEQ ID NO: 34 - protein, 9 aa, Homo sapiens
FAIEALAKA

SEQ ID NO: 35 - protein, 9 aa, Homo sapiens
ILDIAGFEI

SEQ ID NO: 36 - protein, 9 aa, Homo sapiens
FMDGKQACV

SEQ ID NO: 37 - protein, 9 aa, Homo sapiens
VLMIKALEL

SEQ ID NO: 38 - protein, 9 aa, Homo sapiens
KLQVELDNV

SEQ ID NO: 39 - protein, 9 aa, Homo sapiens
KLKDVLLQV

SEQ ID NO: 40 - protein, 10 aa, Homo sapiens
YLLSGAGEHL

SEQ ID NO: 41 - protein, 10 aa, Homo sapiens
FMDGKQACVL

SEQ ID NO: 42 - protein, 10 aa, Homo sapiens
KLRRGDLPFV

SEQ ID NO: 43 - protein, 9 aa, Homo sapiens
QLLQANPIL

SEQ ID NO: 44 - protein, 9 aa, Homo sapiens
MMAKEEELV

SEQ ID NO: 45 - protein, 9 aa, Homo sapiens
QLMAEKLQL

SEQ ID NO: 46 - protein, 9 aa, Homo sapiens
RLEARIAQL

SEQ ID NO: 47 - protein, 9 aa, Homo sapiens
KLQEMEGTV

SEQ ID NO: 48 - protein, 10 aa, Homo sapiens
TLRNTNPNFV

SEQ ID NO: 49 - protein, 10 aa, Homo sapiens
VLDQLRCNGV

SEQ ID NO: 50 - protein, 10 aa, Homo sapiens
NLYRIGQSKV

SEQ ID NO: 51 - protein, 9 aa, Homo sapiens
YIVGANIET

SEQ ID NO: 52 - protein, 9 aa, Homo sapiens
QLEELEDEL

SEQ ID NO: 53 - protein, 10 aa, Homo sapiens
MMQDREDQSI

SEQ ID NO: 54 - protein, 10 aa, Homo sapiens
KLRNWQWWRL

SEQ ID NO: 55 - protein, 10 aa, Homo sapiens
EMMAKEEELV

SEQ ID NO: 56 - protein, 10 aa, Homo sapiens
VLKEGSEYRV

SEQ ID NO: 57 - protein, 10 aa, Homo sapiens
HLSWEWNLSI

SEQ ID NO: 58 - protein, 9 aa, Homo sapiens
EMDKDDESL

SEQ ID NO: 59 - protein, 9 aa, Homo sapiens
TLLGDGPVV

SEQ ID NO: 60 - protein, 9 aa, Homo sapiens
NLAEDIMRL

SEQ ID NO: 61 - protein, 10 aa, Homo sapiens
SLYASSPGGV

SEQ ID NO: 62 - protein, 9 aa, Homo sapiens
RLRSSVPGV

SEQ ID NO: 63 - protein, 9 aa, Homo sapiens
ILLAELEQL

SEQ ID NO: 64 - protein, 9 aa, Homo sapiens
SLQEEIAFL

SEQ ID NO: 65 - protein, 9 aa, Homo sapiens
ALKGTNESL

SEQ ID NO: 66 - protein, 9 aa, Homo sapiens
LQDSVDFSL

SEQ ID NO: 67 - protein, 9 aa, Homo sapiens
SLNLRETNL

SEQ ID NO: 68 - protein, 10 aa, Homo sapiens
FLEQQNKILL

SEQ ID NO: 69 - protein, 10 aa, Homo sapiens
NMKEEMARHL

SEQ ID NO: 70 - protein, 9 aa, Homo sapiens
FMECNLNEL

SEQ ID NO: 71 - protein, 10 aa, Homo sapiens
NLNELVKHGL

SEQ ID NO: 72 - protein, 10 aa, Homo sapiens
HMSEFMECNL

SEQ ID NO: 73 - protein, 10 aa, Homo sapiens
ILHVDNHIGI

SEQ ID NO: 74 - protein, 9 aa, Homo sapiens
GLKSKTHAV

SEQ ID NO: 75 - protein, 9 aa, Homo sapiens
SLIGSKTQI

SEQ ID NO: 76 - protein, 10 aa, Homo sapiens
FMECNLNELV

SEQ ID NO: 77 - protein, 9 aa, Homo sapiens
ALKRAQSEL

SEQ ID NO: 78 - protein, 9 aa, Homo sapiens
GLTADARLL

SEQ ID NO: 79 - protein, 10 aa, Homo sapiens
PLPVSRLVSL

SEQ ID NO: 80 - protein, 9 aa, Homo sapiens
DLTTKNVSI

SEQ ID NO: 81 - protein, 10 aa, Homo sapiens
ELNGKNIEDV

SEQ ID NO: 82 - protein, 9 aa, Homo sapiens
ELAAHQKKI

SEQ ID NO: 83 - protein, 10 aa, Homo sapiens
ELVKHGLRAL

SEQ ID NO: 84 - protein, 10 aa, Homo sapiens
GLKSKTHAVL

SEQ ID NO: 85 - protein, 9 aa, Homo sapiens
GLRALRETL

SEQ ID NO: 86 - protein, 10 aa, Homo sapiens
ELAAHQKKIL

SEQ ID NO: 87 - protein, 9 aa, Homo sapiens
FVFDRPLPV

SEQ ID NO: 88 - protein, 9 aa, Homo sapiens
YVASYLLAA

SEQ ID NO: 89 - protein, 9 aa, Homo sapiens
ILDSVGIEA

SEQ ID NO: 90 - protein, 10 aa, Homo sapiens
YVASYLLAAL